SEQUENCE LISTING (108 sequences)

SEQ ID NO: 1 - protein, 111 aa, Oryctolagus cuniculus
ELVMTQTPSSVSAAVGGTATIDCQSTKSVYNNNALSWYQQKPGQPPKLLIYRASTLTSGVPSRFSGSGSGTQFTLTINDVQCDDAATYYCQGGISGSSDAPFGGGTEVVVK

SEQ ID NO: 2 - protein, 110 aa, Oryctolagus cuniculus
ELVMTQTPSSVSAAVGGTVTINCRASEDIESYLAWYQQKPGQPPKRLIYGASNLASGVSSRFKGSGSGTDFTLTISDLECADAATYYCQCIVGGSTYGNVFGGGTELEIL

SEQ ID NO: 3 - protein, 111 aa, Oryctolagus cuniculus
ELDLTQTPSPVSASVGGTATINCQASQSVYNNYWWAWYQQRPGHPPKLLIYETSKLASGVPSRFKGSGSGTQFTLTIIDVQCDDAATYVCAGGYGTVIDNAFGGGTELEIL

SEQ ID NO: 4 - protein, 111 aa, Oryctolagus cuniculus
ELVMTQTPSPVSAAVGGTVTIACQSSQSVYSGTLLSWYQQKPGQSPKLLIHEASTLASGVPSRFKGSGSGTQFTLTISDLECADAATYYCLGGFDDDADNAFGGGTELEIL

SEQ ID NO: 5 - protein, 109 aa, Oryctolagus cuniculus
ELDLTQTPPSLSASVGGTVTINCQASESISNYLAWYQQKPGQPPKLLIYGASNLESGVPSRFRGSGSGTEFTLTISDMKAEDAATFYCQSGYYTAGDLTFGAGTNVEIK

SEQ ID NO: 6 - protein, 112 aa, Oryctolagus cuniculus
ELVLTQTPASVEAAVGGTVTIKCQASQSIGSYLAWYQQKPGQPPKLLIYGASTLASGVPSRFSGSRSDTQFSLTISDLECADAATYYCQGYYYSDSSSYVNTFGGGTEVVVK

SEQ ID NO: 7 - protein, 109 aa, Oryctolagus cuniculus
ELVMTQTPSSTSAAVGGTVTINCQSSPSVYSNNLAWYQQKPGHSPKLLIYKTSTLASGVPSRFKGSGSGTQFTLTISDLECDDAATYYCQGGYSDFIYGFGGGTELEIL

SEQ ID NO: 8 - protein, 110 aa, Oryctolagus cuniculus
ELVLTQTPSPVSAAVGGTVTINCQASQSVYNNNQLSWFQQKPGQPPKLLIYYASTLASGVPSRFKGSGSGTEYTLTISDVQCDDAATYYCQAYFNGYIWAFGGGTELEIL

SEQ ID NO: 9 - protein, 112 aa, Oryctolagus cuniculus
ELVLTQTPASVSEPVGGTVTIKCQASQSISTALAWYQQKPGQPPRLLIYDVSKLTSGVPSRFSGSGSGTEFTLTISDLECADAATYYCQCSADSSITNYGNAFGGGTELEIL

SEQ ID NO: 10 - protein, 109 aa, Oryctolagus cuniculus
ELVMTQTPSSVSAAVGGTVTIKCQASQNMASSLAWYQQKPGQPPKLLIYDASTLASGVPSRFKGGGSGTQFTLTITDLQCADAATYYCQSDYWTNAFGAFGGGTELEIL

SEQ ID NO: 11 - protein, 110 aa, Oryctolagus cuniculus
ELVLTQTPSPVSAAVGGTVTINCQASQSIGSDLSWYQQKPGQRPKLLIYDASTLASGVPSRFKGSGSGTEFTLTVSGVECADAATYYCQQGYSGTNVDNVFGGGTELEIL

SEQ ID NO: 12 - protein, 113 aa, Oryctolagus cuniculus
ELVLTQTPSSVSAAVGGTVTINCQASQSVWKNNELCWYQQKPGQPPKLLIYKASTLASGVPSRFSGSGSGTQFTLTINDVQCDGAATYYCLGTFNCSSADCNTFGGGTEVVVK

SEQ ID NO: 13 - protein, 110 aa, Oryctolagus cuniculus
ELVMTQTASSVSAAVGGTVTISCQASQSVYNDNWLSWYQQKLGQPPKQLIYRASNLETGVPSRFKGSGSGTQFTLTISDVQCDDAATYYCAGGYSDEVFAFGGGTEVVVK

SEQ ID NO: 14 - protein, 110 aa, Oryctolagus cuniculus
ELVMTQTPSSVSAAVGGTATIDCQSTKSVYNNNALSYQQKPGQPPKLLIYRASTLTSGVPSRFSGSGSGTQFTLTINDVQCDDAATYYCQGGISGSSDAPFGGGTEVVVK

SEQ ID NO: 15 - protein, 109 aa, Oryctolagus cuniculus
ELVLTQTPASVEVAVGGTVTINCQASQSIMNELSWYQQKPGQPPKLLIYRASTLTSGVPSRFSGSGSGTQFTLTINDVQCDDAATYYCQGGISGSSDAPFGGGTEVVVK

SEQ ID NO: 16 - protein, 109 aa, Oryctolagus cuniculus
ELVLTQTPASVEVAVGGTVTINCQASQSIMNELSWYQQKPGQPPKLLIYLASTLASGVPSRFKGSGSGTEFTLTITGVQCDDAATYYCQGGISGSSDAPFGGGTEVVVK

SEQ ID NO: 17 - protein, 111 aa, Oryctolagus cuniculus
ELVMTQTPSSVSAAVGGTATIDCQSTKSVYNNNALSWYQQKPGQPPKLLIYRASTLTSGVPSRFSGSGSGTQFTLTINDVQCDDAATYCCQGGISGSSDAPFGGGTEVVVK

SEQ ID NO: 18 - protein, 112 aa, Oryctolagus cuniculus
ELDLTQTPSSVSAAVGGTVTINCQSSQSVVSNKYLAWYQQKPGQPPMLLIYDVSTLASGVPSRFRGSGSGTQFTLTISDLECDDAATYYCQGSYAGSGYCSTFGGGTELEIL

SEQ ID NO: 19 - protein, 108 aa, Oryctolagus cuniculus
ELDMTQTPASVSEPVGGTVTIKCQASQSISSYLSWYQQKPGQPPKLLIYRASTLESGVPSRFKGSGSGTEYTLTISDLECADAATYYCQIYSSNYGYAFGGGTELEIL

SEQ ID NO: 20 - protein, 112 aa, Oryctolagus cuniculus
ELVLTQTPASVEAAVGGTVTIKCQASQSISSYLAWYQQKPGQPPKLLIYRASTLASGVPSRFKGSGSGTEFTLTISDLECADAATYYCQSYYGSSTTTYGNPFGGGTELEIL

SEQ ID NO: 21 - protein, 109 aa, Oryctolagus cuniculus
ELDLTQTPSSVSAAVGGTVTINCQASESISNYLAWYQQKPGQPPKLLIYGASNLESGVPSRFRGSGSGTEFTLTISDMKAEDAATFYCQSGYYTAGDLTFGAGTNVEIK

SEQ ID NO: 22 - protein, 107 aa, Oryctolagus cuniculus
ELDLTQTPPSLSASVGGTVTINCLASENVYSAVAWYQQKPEKPPTLLISGASNLESGVPPRFSGSGSGTDYTLTIGGVQAEDAATYFCQGYSSYPPTFGAGTNVEIK

SEQ ID NO: 23 - protein, 111 aa, Oryctolagus cuniculus
ELVLTQTPSSVSAAVGGTVSISCQASQSVYKNNYLSWFQQKPGQPPKLLIYDASTLASGVPSRFSGSGSGTEFTLTISDLECADAATYYCLGDDSSSSENVFGGGTELEIL

SEQ ID NO: 24 - protein, 111 aa, Oryctolagus cuniculus
ELVLTQTPSPVSAAVGGTVTISCQSSQSVYNNNYLSWFQQKPGQPPKLLIYETSKLESGVPSRFSGSGSGTQFTLTISDVQCDDAATYYCAGVYTTASDDSFGGGTEVVVK

SEQ ID NO: 25 - protein, 109 aa, Oryctolagus cuniculus
ELVMTQTPSSVEAPVGGTITIKCQASEDIYRLLAWYQQKPGQPPKLLIYDASTLASGVSSRFKGSGSGTEFTLTISDLECADGATYYCQSYYANSIGNAFGGGTEVVVK

SEQ ID NO: 26 - protein, 109 aa, Oryctolagus cuniculus
ELDLTQTPASVEAAVGGTVTINCRASEDIESLLAWYQQKPGQRPKLLIYDTSTLESGVPSRFKGSGSGTEYTLTISDLECDDAATYYCQSASYSSKSNPFGGGTELEIL

SEQ ID NO: 27 - protein, 111 aa, Oryctolagus cuniculus
ELVLTQTPSPVSAAVGGTVSISCQSSKNVYNNNYLAWYQQKPGQPPKLLIYTASSLASGVPSRFKGSGSGTQFSLTISDLECDDAATYYCAGGTGSTGDTAFGGGTEVVVK

SEQ ID NO: 28 - protein, 114 aa, Oryctolagus cuniculus
ELVMTQTPASVSEPVRGTVTIKCQASQNIRSWLSWYQQKPGQPPKLLIYQASKLASGVPSRFKGSGSETDFTLTISDLECADAATYYCQSNYDSSTTISYGADTFGGGTEVVVK

SEQ ID NO: 29 - protein, 113 aa, Oryctolagus cuniculus
ELVMTQTPASVSAAVGGTVTIKCQASQSVYGNNLLSWYQQKPGQPPKLLIYKASKLASGVPSRFSGSGSGTEFTLTISGVQCDDAATYYCLGVDYYQHNDGAAFGGGTEVVVK

SEQ ID NO: 30 - protein, 111 aa, Oryctolagus cuniculus
ELVLTQTPSPVSAAVGGTASISCQSSVSVYDNDRLSWYQQKPGQRPKLLIYAVSALASGVPSRFKGSGYGTQFTLTISDVQCDDAATYYCAGGYNDGFDGTFGGGTELEIL

SEQ ID NO: 31 - protein, 110 aa, Oryctolagus cuniculus
ELVLTQTPASMEVPVGGTVTITCQASESISSYLNWYQQKPGQPPKLLIFQASKLASGVPSRFKGSGSGTEFTLTISDVQCDDAATYYCQQGLINSNIDNTFGGGTEVVVK

SEQ ID NO: 32 - protein, 112 aa, Oryctolagus cuniculus
ELVLTQTPASVEVAVGGTVTINCQASQSIMNELSWYQQKPGQPPKLLIYLASTLASGVPSRFKGSGSGTEFTLTITGVQCDDAATYYCQQGYSHSNGAIDNVFGGGTELEIL

SEQ ID NO: 33 - protein, 112 aa, Oryctolagus cuniculus
ELDLTQTPASVEVAVGGTVTINCQASEDIGNLLAWYQQKPGQPPKLLIYKASTVASGVSSRFKGSGSGTQFTLTISGVECDDAATYYCQNNYGSSTIGHVYDFGGGTEVVVK

SEQ ID NO: 34 - protein, 111 aa, Oryctolagus cuniculus
ELVMTQTPASVEAAVGGTVTIKCQASQSIGTALAWYQQKPGQPPKLLIYKASTLASGVPSRFKGSGSGTQFTLTISDLECADAATYYCQGYDIGSGYGSNPFGGGTEVVVK

SEQ ID NO: 35 - protein, 110 aa, Oryctolagus cuniculus
ELDLTQTPASVEVPVGGTVTIKCQASQSISSELSWYQQQPGQRPKLLIYTAANLASGVPSRFKGSRSGTEFTLTISDLECADAATYYCQQGYSGDNVDNSFGGGTELEIL

SEQ ID NO: 36 - protein, 109 aa, Oryctolagus cuniculus
ELDLTQTPASVSEPVGGTVTIKCQASESVQNYLAWYQQKPGQPPKLLIYRASNLESGVSSRFKGSRSGTEFTLTISDLECADAATYYCQCTAGGNGYVGFGGGTELEIL

SEQ ID NO: 37 - protein, 112 aa, Oryctolagus cuniculus
ELVMTQTPASVSAAVGGTVTINCQASQSVYSNNYLSWYQQKPGQPPKLLIYKASTLASGVPSRFKGSGSGTQFTLTISDLECDDAATYYCAGYKSWNNDDFGFGGGTEVVVK

SEQ ID NO: 38 - protein, 110 aa, Oryctolagus cuniculus
ELVLTQTPASVSEPVGGTVTIKCQASQSINSWLSWYQQKPGQPPKLLIYQASKLASGVPSRFKGSGSGTEFTLTISDLECADAATYYCQNYYGISSYGRAFGGGTEVVVK

SEQ ID NO: 39 - protein, 111 aa, Oryctolagus cuniculus
ELVMTQTPSPVSAAVGGTVTISCQSSQSVDNNNRLAWYQQKVGQPPKLLIYKASTLASGVPSRFKGSGSGTQFTLTISDLECDDAATYYCAGDYKGNGDNDFGGGTELEIL

SEQ ID NO: 40 - protein, 108 aa, Oryctolagus cuniculus
ELVMTQTPASVSEPVGGTVTIKCQASQSIINLLAWYQQKPGQRPRLLMYSAFTLASGVPSRFKGSGSETEYTLTISDLECADAATYYCQSYYGGTYIAFGGGTELEIL

SEQ ID NO: 41 - protein, 112 aa, Oryctolagus cuniculus
ELVLTQTPSSKSVPVGDTVTINCQASETVYDNNRLVWFQQKPGQPPKLLIYKASTLASGVPSRFKGSGSGTQFTLTISDVVCDDAATYYCAGYKSRGVDGSDFGGGTELEIL

SEQ ID NO: 42 - protein, 112 aa, Oryctolagus cuniculus
ELDLTQTPPSVSAAVGGTLTISCQSSESVYSNNRLSWYQQKPGQPPKLLIYEASTLSSGVPSRFKGSGSGTQFTLTISDVVCDDAATYYCQGNYYSSDWYNSFGGGTELEIL

SEQ ID NO: 43 - protein, 107 aa, Oryctolagus cuniculus
ELDLTQTPSSVSAAVGGTVTIKCQASQSVSSYLTWYQQKPGQPPKLLIYGASNLESGVPSRFRGSGSGTQFTLTISGMKAEDVATYYCHQHNSYPLTFGAGTNVEIK

SEQ ID NO: 44 - protein, 111 aa, Oryctolagus cuniculus
ELVMTQTPSPVSAAMGGTVSISCQSSKSVANNNELSWYQQKPGQPPKLLIYEASKLASGVPSRFKGSGSGTQFTLTISDVQCDDAATYYCLGDYYSSSDNAFGGGTEVVVK

SEQ ID NO: 45 - protein, 107 aa, Oryctolagus cuniculus
ELVLTQTPSSVSAAVGGTVTINCQASQSISNLLAWYQQKPGQPPKLLIYEASNLESGVPSRFRGSGSGTEFTLTISGMKAEDAATYYCQSGYYSALTFGAGTNVEIK

SEQ ID NO: 46 - protein, 111 aa, Oryctolagus cuniculus
  SITE 4: note = misc_feature - Xaa can be any naturally occurring amino acid
  SITE 6: note = misc_feature - Xaa can be any naturally occurring amino acid
ELVXMXTQTPASVSAAVGGTVSISCQASQSVDWTTNLAWYQQKPGQRPKLLIYKASTLASGVPSRFSGSGSGTQFTLTISGVQCDDAATYYCAGAATNTWAFGGGTELEIL

SEQ ID NO: 47 - protein, 113 aa, Oryctolagus cuniculus
ELVLTQTPASVEAAVGGTVTINCQASQSVYNNKNLAWYQQKPGQPPKLLIYRASTLASGVPSRFSGSGSGTQFTLTISGAQCDDAATYYCQGEFSCSSGDCIAFGGGTEVVVK

SEQ ID NO: 48 - protein, 113 aa, Oryctolagus cuniculus
ELVMTQTPASVSEPVGGTVTIKCQASQSIGNALAWYQQKPGQPPKLLIYGASNLESGVPSRFKGSGSGTEFTLTISDLECADAATYYCQSYYYSSSSSYGSYAFGGGTEVVVK

SEQ ID NO: 49 - protein, 110 aa, Oryctolagus cuniculus
ELDMTQTPASVSAAVGGTVTIKCQASQTIGNYLAWYQQKPGQPPKLLISQASTLASGVPSRFRGSGSGTQFTLTISGVQCADAATYYCQCTDYGDTFGNAFGGGTEVVVK

SEQ ID NO: 50 - protein, 110 aa, Oryctolagus cuniculus
ELVMTQTPASVSEAVGGTVTIKCQASQSIGGWLAWYQQKPGQRPKLLIYGASNLASGVPSRFSGSGSGTEFTLTISGVQCDDAATYYCQHGYTDTNIDNSFGGGTEVVVK

SEQ ID NO: 51 - protein, 111 aa, Oryctolagus cuniculus
ELDMTQTPASVSEPVGGTVTIKCQASEGIYNNLVWYQQRPGQPPKLLIYRASTLASGVPSRFSGSGSGTHFTLTISDLECADAATYYCQSYYIRGSDFVNAFGGGTEVVVK

SEQ ID NO: 52 - protein, 111 aa, Oryctolagus cuniculus
ELVMTQTPSSASEPVGGTVTIKCQASESISSRLAWYQQKPGQRPKLLIYGASNLESGVPSRFKGSGSGTEYSLTISDLECDDAATYYCQSTYVSSTADTFAFGGGTEVVVK

SEQ ID NO: 53 - protein, 110 aa, Oryctolagus cuniculus
ELDLTQTPASVEAAVGGTITINCQASESIDSSCASWYQQKPGQPPKLLIYKASTLASGVPSRFKGSGSGTQFSLTISGVRCDDAATYYCSGYKSYSNDDNGFGGGTEVVV

SEQ ID NO: 54 - protein, 109 aa, Oryctolagus cuniculus
ELDMTQTPSSVSAAVGGTVTINCQASQSIGSWLAWYQQKPGQPPKQLIYDASTLASGVPSRFKGSGSGTEFTLTISGVQCDDAATYYCAGTYEISGWCGAFGGGTERRS

SEQ ID NO: 55 - protein, 109 aa, Oryctolagus cuniculus
ELDMTQTPASVEAAVGGTVTIKCQASQNIYSNLAWYQQKPGQPPKLLIYEASRLASGVPSRFSGSGSGTQFTLTISGVQCDDAATYYCLGAYNDDSDNTFGGGTELEIL

SEQ ID NO: 56 - protein, 111 aa, Oryctolagus cuniculus
  SITE 14: note = misc_feature - Xaa can be any naturally occurring amino acid
  SITE 96: note = misc_feature - Xaa can be any naturally occurring amino acid
ELDMTQTPSSTSEXVGGTVTIKCQASQNIGSDLSWYQQKPGQPPKLLIYQASKLASGVPSRFSGSRSRTEYTLAISGVQCDDAVTYYCLGVYGYSXEDGAAFGGGTELEIL

SEQ ID NO: 57 - protein, 111 aa, Oryctolagus cuniculus
ELDMTQTPSSTSEPVGGTVTIKCQASQNIGSDLSWYQQKPGQPPKLLIYQASKLASGVPSRFSGSRSRTEYTLAISGVQCDDAVTYYCLGVYGYSSEDGAAFGGGTELEIL

SEQ ID NO: 58 - protein, 111 aa, Oryctolagus cuniculus
ELDMTQTPASVSEPVGGTVTISCQSSQSVFDSNELSWYQQKPGQRPKPLIYDASKLASGVPSRFKGSGSGTRFTLTISDVQCDDAATYYCAGAFTTSSDNLFGGGTELEIL

SEQ ID NO: 59 - protein, 114 aa, Oryctolagus cuniculus
ELVMTQTPASVEAAVGGTVTINCQASQSVYNNNRLAWYQQKPGQPPKLLIYYASDLASGVSSRFKGSGSGTQFTLTISDLECADAATYYCQTSYWRSSADTSAVFGGGTELEIL

SEQ ID NO: 60 - protein, 111 aa, Oryctolagus cuniculus
ELVMTQTPPSLSASVGETVRIRCLASEDIYSGISWYQQKPGKPPTLLIYGASNLESGVPPRFSGSGSGTDYTLTIGGVQAEDAATYYCLGGYSYSSTGSLTFGAGTNVEIK

SEQ ID NO: 61 - protein, 110 aa, Oryctolagus cuniculus
  SITE 12: note = misc_feature - Xaa can be any naturally occurring amino acid
ELVLTQTPPSLXASVGETVRIRCLASEDIYSGISWYQQKPGKPPTLLISGAANLASGVPSRFSGSGSGTDYTLTIGGVQAEDAATYYCLGGWSYSDSGTTFGAGTNVEIK

SEQ ID NO: 62 - protein, 109 aa, Oryctolagus cuniculus
ELVMTQTPSSVSAAVGGTVTINCQASKNINNYLAWYQQKPGQPPKLLIYGASNLESGVPSRFRGSGYGTEFTLTISDMKAEDAATYVCQSAYYSGGDLTFGAGTNVEIK

SEQ ID NO: 63 - protein, 107 aa, Oryctolagus cuniculus
ELVMTQTPSSVSAAVGGTVTINCQASQSISNLLAWYQQKPGQPPKLLIYEASNLESGVPSRFRGSGSGTEFTLTISGMKAEDAATYYCQSGYYSALTFGAGTNVEIK

SEQ ID NO: 64 - protein, 107 aa, Oryctolagus cuniculus
  SITE 76: note = misc_feature - Xaa can be any naturally occurring amino acid
ELVLTQTPSSVSAAVGGTVTINCQASQSISNLLAWYQQKPGQPPKLLIYEASNLESGVPSRFRGSGSGTEFTLTIXGMNAEDAATYYCQSGYYSALTFGAGTNVEIK

SEQ ID NO: 65 - protein, 109 aa, Oryctolagus cuniculus
ELVMTQTPSPVSAAVGGTVTIKCQSSQSVYNNNLLSWYQQKPGQPPKLLIYDASNLASGVPDRFSGSGSGTQFTLTISGVQCDDAATYYCLGGYDDDAEFGVGTNVEIK

SEQ ID NO: 66 - protein, 111 aa, Oryctolagus cuniculus
  SITE 4: note = misc_feature - Xaa can be any naturally occurring amino acid
ELVXTQTASPVSAAVGGTVTINCQSSQSVVDNNRLAWYQQKPGQPPKLLIYGASTLASGVPSRFKGSGSGTDFTLTISDVQCDDAATYYCLGTYVDDSHDAFGGGTEVVVK

SEQ ID NO: 67 - protein, 111 aa, Oryctolagus cuniculus
ELVMTQTPSPVSAAVGGTVTISCQASQSVYSNYLSWFQQKPGQPPKLLIYGASTLASGVPSRFKGSGSGTQFTLTISDVQCDDAATYYCAGGYATSSDNRAFGGGTEVVVK

SEQ ID NO: 68 - protein, 112 aa, Oryctolagus cuniculus
ELVMTQTPSPVSAAVGGTVSINCQSSPSVYGSYLSWYQQKPGQPPKLLIYYASTLASGVPSRFKGSGSGTQFTLTISDVQCDDGATYYCAGGYSSGSDTGSAFGGGTEVVVK

SEQ ID NO: 69 - protein, 111 aa, Oryctolagus cuniculus
ELVLTQTPSPVSAAVGGTVSISCQSSKSVYNNNWLSWFQQKPGQPPKQLIYYASTLASGVPSRFKGSGSGTQFTLTISDVQCDDAATYYCAGGYSSSSDNLFGGGTELEIL

SEQ ID NO: 70 - protein, 111 aa, Oryctolagus cuniculus
ELDLTQTPSSTSEPVGGTVTIKCQASQSIGTKLSWYQQKPGQPPKLLIYQASKLASGVPSRFSGSGSGTDFTLTISDVQCDDAATYYWLGSYDCRSADCGAFGGGTEVVVK

SEQ ID NO: 71 - protein, 107 aa, Oryctolagus cuniculus
ELVLTQTPSSVSAAVGGTVTINCQASQSISNLLAWYQQKPGQPPKLLIYEASNLESGVPSRFRGSGSGTEFTLTISGMKAEDAATYYCQSGYYSALTFGAGTNVDIK

SEQ ID NO: 72 - protein, 107 aa, Oryctolagus cuniculus
ELDMTQTPSSVSAAVGGTVTINCQASQSISNLLAWYQQKPGQPPKLLIYEASNLESGVPSRFRGSGSGTEFTLTISGMKAEDAATYYCQSGYYSALTFGAGTNVEIK

SEQ ID NO: 73 - protein, 107 aa, Oryctolagus cuniculus
ELDMTQTPSSVSAAVGGTVTINCQASQSISNLLAWYQQKPGQLPKLLIYEASNLESGVPSRFRGSGSGTEFTLTISGMKAEDAATYYCQSGYYSALTFGAGTNVEIK

SEQ ID NO: 74 - protein, 107 aa, Oryctolagus cuniculus
ELDLTQTPSSTSEPVGGTVTINCQASQSISNLLAWYQQKPGQPPKLLIYEASNLESGVPSRFRGSGSGTEFTLTISGMKAEDAATYYCQSGYYSALTFGAGTNVEIK

SEQ ID NO: 75 - protein, 107 aa, Oryctolagus cuniculus
ELVLTQTPSSVSAAVGGTVTINCQASQSISNLLAWYQQKPGQPPKLLIYEASNLESGVPSRFRGSGSGTEFTLTISGMKAEDAATYYWQSGYYSALTFGAGTNVEIK

SEQ ID NO: 76 - protein, 107 aa, Oryctolagus cuniculus
ELVMTQTPASVSEPVGGTVTIKCQASQSIGNALAWYQQKPGQPPKLLIYGASNLESGVPSRFRGSGSGTEFTLTISGMKAEDAATYYCQSGYYSALTFGAGTNVEIK

SEQ ID NO: 77 - protein, 111 aa, Oryctolagus cuniculus
ELVLTQTASPVSAAVGGTVTINCQSSQSVVDNNRLAWYQQKPGQPPKLLIYGASTLASGVPSRFKGSGSGTDFTLTISDVQCDDAATYYCLGTYVDDSHDAFGGGTEVVVK

SEQ ID NO: 78 - protein, 107 aa, Oryctolagus cuniculus
ELVMTQTPASVSEPVGGTVTIKCQASQSIGNALAWYQQKPGQPPKLLIYEASNLESGVPSRFRGSGSGTEFTLTISGMKAEDAATYYCQSGYYSALTFGAGTNVEIK

SEQ ID NO: 79 - protein, 107 aa, Oryctolagus cuniculus
ELVLTQTPSSVSAAVGGTVTINCQASQSISNLLAWYQQKPGQPPKLLIYEASNLESGVPSRFRGSGSGTEFTLTISGMKGEDAATYYCQSGYYSALTFGAGTNVEIK

SEQ ID NO: 80 - protein, 107 aa, Oryctolagus cuniculus
ELVLTQTPSSVSAAVGGTVTINCQASQSISNLLAWYQQKPGQPPKLLIYEASNLESGVPSRFRGSGSGTEFTLTISGMKAEDAATYYCQSGYYSALTFGAGTNVESK

SEQ ID NO: 81 - protein, 107 aa, Oryctolagus cuniculus
ELDLTQTPSSTSEPVGGTVTIKCQASQSIGTKLSWYQQKPGQPPKLLIYEASNLESGVPSRFRGSGSGTEFTLTISGMKAEDAATYYCQSGYYSALTFGAGTNVEIK

SEQ ID NO: 82 - protein, 111 aa, Oryctolagus cuniculus; identical to SEQ ID NO: 44

SEQ ID NO: 83 - protein, 107 aa, Oryctolagus cuniculus; identical to SEQ ID NO: 63

SEQ ID NO: 84 - protein, 111 aa, Oryctolagus cuniculus; identical to SEQ ID NO: 57

SEQ ID NO: 85 - protein, 107 aa, Oryctolagus cuniculus
ELDLTQTPSSVSAAVGGTVTINCQASQSISNLLAWYQQKPGQPPKLLIYEASNLESGVPSRFRGSGSGTEFTLTISGMKAEDAATYYCQSGYYSALTFGAGTNVEIK

SEQ ID NO: 86 - protein, 113 aa, Oryctolagus cuniculus; identical to SEQ ID NO: 48

SEQ ID NO: 87 - protein, 110 aa, Oryctolagus cuniculus
ELVMTQTPASVEAAVGGTVTIKCQASENIYSQLSWYQQKPGQRPKLLIYYASNLASGVSSRFTGSGSGTEYTLTISDLECADAATYYCQQGYTAGDVDNAFGGGTEVVVK

SEQ ID NO: 88 - protein, 111 aa, Oryctolagus cuniculus
ELDLTQTPSSTSEPVGGTVTIKCQASQSIGTKLSWYQQKPGQPPKLLIYQASKLASGVPSRFSGSGSGTDFTLTISDVQCDDAATYYCLGSYDCRSADCGAFGGGTEVVVK

SEQ ID NO: 89 - protein, 107 aa, Oryctolagus cuniculus; identical to SEQ ID NO: 45

SEQ ID NO: 90 - protein, 109 aa, Oryctolagus cuniculus
ELVLTQTASPVSAAVGGTVTINCQSSQSVVDNNRLAWYQQKPGQPPKLLIYEASNLESGVPSRFRGSGSGTEFTLTISGMKAEDAATYYCQSGYYSALTFGAGTNVEIK

SEQ ID NO: 91 - protein, 107 aa, Oryctolagus cuniculus
ELVLTQTPSSVSAAVGGTVTINCQASQSISNLLAWYQQKPGQPPKLLIYEASNLESGVPSRFRGSGSGTQFTLTISGMKAEDAATYYCQSGYYSALTFGAGTNVEIK

SEQ ID NO: 92 - protein, 109 aa, Oryctolagus cuniculus
ELVMTQTPSPVSAAMGGTVSISCQSSKSVANNNELSWYQQKPGQPPKLLIYEASNLESGVPSRFRGSGSGTEFTLTISGMKAEDAATYYCQSGYYSALTFGAGTNVEIK

SEQ ID NO: 93 - protein, 107 aa, Oryctolagus cuniculus
ELVMTQTPSSVSAAVGGTVTINCQASQSISNLLAWYQQKPGQPPKLLIYEASNLESGVPSRFRGSGSGTQFTLTISGMKAEDAATYYCQSGYYSALTFGAGTNVEIK

SEQ ID NO: 94 - protein, 112 aa, Oryctolagus cuniculus
ELVMTQTPASVSAAVGGTVTINCQASQSLYNNKNLAWYQQKPGQPPKRLMYATSNLASGVSSRFKGSGSGTQFTLTISDLECADAATYYCQSPFYGSGDVFAFGGGTELEIL

SEQ ID NO: 95 - protein, 113 aa, Oryctolagus cuniculus; identical to SEQ ID NO: 48

SEQ ID NO: 96 - protein, 109 aa, Oryctolagus cuniculus
ELVMTQTPASVSEPVGGTVTINCQASQSIGSNLAWYQQKAGQPPKLLIYYASTLESGVPSRFSGSGSGTQFTLTISDVQCDDAATYYCAGADTSSADSAFGGGTELEIL

SEQ ID NO: 97 - protein, 111 aa, Oryctolagus cuniculus
ELVLTQPQSVSGSLGQTVSISCNRDSGNIEDYYVHWYQQHPGKAPTTVVYNDDQRPSGVPDRFSGSIDSTSNSASLTITDLLAEDEADYYCLSSDSSANVFGGGTQLTVTG

SEQ ID NO: 98 - protein, 106 aa, Oryctolagus cuniculus
LDMTQTPSSVSAAVGGTVTINCQASQSISNLLAWYQQKPGQPPKLLIYEASNLESGVPSRFRGSGSGTEFTLTISGMKAEDAATYYCQSGYYSALTFGAGTNVEIK

SEQ ID NO: 99 - protein, 113 aa, Oryctolagus cuniculus
ELVMTQTPASVEAAVGGTVTINCQASQSVYNNKNLAWYQQKPGQPPKLLIYRASTLASGVPSRFSGSGSGTQFTLTISGAQCDDAATYYCQGEFSCSSGDCIAFGGGTEVVVK

SEQ ID NO: 100 - protein, 112 aa, Oryctolagus cuniculus
ELDMTQTPSSTSEPVGGTVTIKCQASQNIGSDLSWYQQKPGQPPKLLIYQASKLASGVPSRFSGSRSRTEYTLAISGVQCDDAVTYYCLGVYGYSSEDGAAFGGGTELEILG

SEQ ID NO: 101 - protein, 109 aa, Oryctolagus cuniculus
ELVMTQTPASVSAAVGGTVSISCQASQSVDWTTNLAWYQQKPGQRPKLLIYKASTLASGVPSRFSGSGSGTQFTLTISGVQCDDAATYYCAGAATNTWAFGGGTELEIL

SEQ ID NO: 102 - protein, 111 aa, Oryctolagus cuniculus
ELDLTQTPASVEAAVGGTITINCQASESIDSSCASWYQQKPGQPPKLLIYKASTLASGVPSRFKGSGSGTQFSLTISGVRCDDAATYYCSGYKSYSNDDNGFGGGTEVVVK

SEQ ID NO: 103 - protein, 108 aa, Oryctolagus cuniculus
ELVLTQTPSSVSAAVGGTVTINCQASQSISNLLAWYQQKPGQPPKLLIYDASNLESGVPSRFRGSGSGTEFTLTISGMKAEDAATYYCQSGYYSALTFGAGTNVEIKG

SEQ ID NO: 104 - protein, 107 aa, Oryctolagus cuniculus
ELDLTQTPSSVSAAVGGTVTINCQASQSISNLLAWYQQKPGQPPKLLIYEASNLESGVPSRFRGSGSGTQFTLTISGMKAEDAATYYCQSGYYSALTFGAGTNVEIK

SEQ ID NO: 105 - protein, 111 aa, Oryctolagus cuniculus; identical to SEQ ID NO: 77

SEQ ID NO: 106 - protein, 111 aa, Oryctolagus cuniculus; identical to SEQ ID NO: 44

SEQ ID NO: 107 - protein, 107 aa, Oryctolagus cuniculus; identical to SEQ ID NO: 63

SEQ ID NO: 108 - protein, 107 aa, Oryctolagus cuniculus; identical to SEQ ID NO: 85

(19) United States
(12) Patent Application Publication
(10) Pub. No.: US 2024/0245800 A1
(43) Pub. Date: Jul. 25, 2024

(54) HIGHLY SPECIFIC RABBIT SINGLE -DOMAIN ANTIBODIES FOR DRUG DELIVERY IN IMMUNOTHERAPY APPLICATIONS

(71) Applicants: TECHNOPHAGE, INVESTIGAÇÃO E DESENVOLVIMENTO EM BIOTECNOLOGIA, S.A., Lisboa (PT); FACULDADE DE MEDICINA VETERINÁRIA DA UNIVERSIDADE DE LISBOA, Lisboa (PT)

(72) Inventors: Frederico Nuno Castanheira Aires Da Silva, Lisboa (PT); Joana Nunes Ribeiro Dias Gomes, Lisboa (PT); Ana Filipa Santos André, Alvor (PT); Sandra Isabel Rodrigues De Aguiar, Lisboa (PT); Soraia Rafaela Santiago De Oliveira, Fernão Ferro (PT); Luís Manuel Morgado Tavares, Linda-a-Velha (PT)

(21) Appl. No.: 18/577,237

(22) PCT Filed: Jul. 7, 2022
(86) PCT No.: PCT/IB2022/056303

§ 371 Date: Jan. 5, 2024

(30) Foreign Application Priority Data
Jul. 7, 2021 (PT) 117330
Sep. 13, 2021 (PT) 117461

(51) Int. Cl. A61K 47/68; A61K 47/69; C07K 16/30

(52) U.S. Cl. CPC A61K 47/6867; A61K 47/6913; C07K 16/30; C07K 2317/10; C07K 2317/569; C07K 2317/77

(57) ABSTRACT

The present disclosure relates to the development of drug delivery systems comprising single domain antibodies (sdAbs). For this purpose, antibody-drug conjugates (ADCs) are provided with high selectivity and efficiency to be used advantageously in cancer therapy, and a drug-delivery system targeting the central nervous system (CNS) pathologies. The ADC molecules developed for therapy, namely for cancer therapy are obtained from rabbit derived sdAbs comprising a potent cytotoxic payload, a SN38 small molecule conjugated with the free exposed cysteine at position 80, 23 or 88 of the V_{L} framework. The drug delivery systems developed targeting the BBB endothelial cell receptors of the central nervous system (CNS) comprise rabbit derived single-domain antibodies (sdAb) conjugated at the surface of liposomes encapsulated with a suitable drug enabling an efficient blood-brain barrier (BBB) translocation. The respective process of production is also herein disclosed. Therefore, the present disclosure is in the domain of genetic engineering, biotechnology, pharmaceuticals and medicine.

### TECHNICAL DOMAIN

The present invention relates to the development of drug delivery systems comprising single domain antibodies (sdAbs).

For this purpose, antibody-drug conjugates (ADCs) are provided with high selectivity and efficiency to be used advantageously in cancer therapy, and drugs targeting the central nervous system (CNS) pathologies.

The ADC molecules developed for therapy, namely for cancer therapy are obtained from rabbit derived sdAbs comprising a potent cytotoxic payload, a SN38 small molecule conjugated, with the free exposed cysteine at position 80 of the V_{L} framework.

It is also disclosed the process to obtain such antibody fragments and their use as medicaments. The process of obtaining such molecule includes the selective conjugation of a free cysteine present in rabbit derived sdAbs with a chemical payload, without requiring further genetic engineering manipulation.

The drug delivery systems developed targeting the BBB endothelial cell receptors of the central nervous system (CNS) comprise rabbit derived single-domain antibodies (sdAb) conjugated at the surface of liposomes encapsulated with a suitable drug enabling an efficient blood-brain barrier (BBB) translocation. The respective process of production is also herein disclosed.

Therefore, the present invention is in the domain of genetic engineering, biotechnology, pharmaceuticals and medicine.

### PRIOR ART

Responsible for almost 10 million deaths and 19.3 million new cases in 2020 alone, cancer incidence and mortality continue to grow worldwide. Owing to the rapid growth and aging of the population, as well as the increasing prevalence of risk factors, the number of new cancer cases is expected to rise by 47% over the next 20 years. As such, despite the remarkable progress made in cancer treatment over the last decades, there is still a great demand for new solutions.

Almost 80 years have passed since the advent of the modern era of cancer chemotherapy, nonetheless, conventional chemotherapy remains mostly unchanged, still resorting to cytotoxic drugs that target the cell cycle of rapidly dividing cancer cells. Even though it is associated with significant clinical disadvantages, including a narrow therapeutic window, increasing drug resistance, and non-specific toxicity, cytotoxic chemotherapy continues to be at the core of cancer treatment.

In the 1970's, the emergence of monoclonal antibodies (mAbs)-based therapies promised to revolutionize cancer treatment. By specifically targeting cancer cells, mAbs could reduce non-specific toxicities, and directly promote signalling-induced death or mediate an anti-tumour immune response. To date, approximately 30 mAbs have been approved for cancer treatment by the US Food and Drug Administration (FDA), however, most mAbs do not possess clinical efficacy as single agents and are currently used in combination with conventional cytotoxic regimes.

The advance of mAb technology over the following decades allowed conjugating antibodies with a diversity of antitumour effector molecules (e.g., cytotoxic drugs, radiopharmaceuticals, and immunotoxins), propelling the development of mAb-based targeted therapies and immunotherapies, including an emerging novel class of anticancer treatment agents called antibody-drug conjugates (ADCs). ADCs combine the tumour selectivity, pharmacokinetics and biodistribution properties of antibodies with the cytotoxic potency of small molecules. This concept of selective delivery was first envisioned by Paul Ehrlich in the early 20th century, who reasoned the “magic bullet” theory, and revisited when mAbs were considered suitable moieties for the creation of such magic bullets.

Nevertheless, the journey taken for the development of an effective ADC revealed itself to be long and remarkably challenging. The first generation of ADCs, consisting of a mAb conjugated with a conventional chemotherapeutic agent, had limited success due to low potency and/or toxicity associated with ADC instability and systemic loss of the drug. Thus, the next generations of ADCs resorted to more potent payloads, relied on humanized and human mAbs to reduce immunogenicity, while optimized linker stability and intracellular release, increased target and antibody selectivity. This led to FDA approval of the first ADC in 2000, gemtuzumab ozogamicin, for the treatment of CD33-expressing acute myeloid leukemia (AML).

Following that, an impressive expansion of the clinical ADC pipeline took place with more than eighty ADCs enrolling in approximately six hundred clinical trials to date, at different clinical stages. However, since 2011 only nine additional ADCs were granted FDA approval. Over the years, several ADCs showing great potential in early pre-clinical stages, have failed to progress or were even abruptly terminated. Approximately 55 ADC clinical trials have been cancelled so far, many due to lack of efficacy and off-target cytotoxicity. As such, a careful and critical re-evaluation of preclinical and clinical results is essential to inform future trials and allow the success of this promising platform.

One of the main challenges affecting ADCs, including those already on the market, is the heterogeneous composition of generated products, which means that each mAb is linked to a variable number of cytotoxic drugs in different locations. This heterogeneity leads to a different drug-to-antibody ratio (DAR), generating products with variable pharmacokinetics and therapeutics profiles. This problem is mostly associated with conventional drug bioconjugation methods that couple antibodies through either surface-exposed lysines (˜70 to 90) or cysteines from interchain disulphides (8 in IgG1), two abundant features in IgG mAbs.

Moreover, conserved cysteines play a fundamental role in the antibody structure and its use in conjugation often leads to aggregation issues and improper folding. New approaches have been used to overcome these drawbacks, including site-specific conjugation methods that have resulted in a new generation of more uniform, molecularly defined ADCs. Yet, most of these methodologies are not compatible with the scale-up of the manufacturing process required for ADC production.

Additionally, most ADCs currently in development and on the market consist of a complete IgG antibody. However, the clinical use of these IgG based moieties has been hampered by the low penetration in tumour tissues, as a consequence of their large molecular weight, and by the high manufacturing costs in mammalian cells. Moreover, there is now evidence that the Fc domain of an IgG may be redundant or even unfavorable for ADCs efficacy. In fact, ADCs prolonged half-life promoted by the FcRn increases exposure to healthy tissues, while FcγR cross-react with endothelial and immune cells, both of which are biological processes related to off-target toxicity.

Therefore, further improvements to ADC design and development are required to allow the synthesis of more homogeneous and stable molecules with higher therapeutic indexes.

To overcome these problems, the present invention provides drug delivery systems based on single domain derived rabbit antibodies (sdAbs). sdAbs are presently the smallest functional antibody fragments, only consisting of a VH or VL unit. These small size scaffolds of about 15 kDa possess higher tumour penetration and accessibility to targets not easily reached by large size conventional mAbs.

As mentioned before, a second aspect of the present invention is to provide a drug delivery system targeting the central nervous system (CNS) pathologies, namely that can cross the blood-brain barrier (BBB), based on rabbit derived single-domain antibody (sdAb) towards BBB endothelial cell receptors.

Despite the major advances in the fields of neuroscience and drug development, the efficacy of many potential therapeutics for treatment of central nervous system (CNS) diseases has been systematically challenged by the low permeability of the blood-brain barrier (BBB). In fact, this physical and metabolic selective barrier between the brain and the systemic circulation, is the main bottleneck in brain-targeted drug development and the most important factor limiting the treatment of major unmet neurodegenerative disorders such as Alzheimer's, Parkinson's disease and brain tumours. To overcome this barrier, several strategies have been investigated in the past two decades. One of such approaches is the development of specific antibodies that target endogenous BBB transport mechanisms, such as the receptor-mediated transcytosis (RMT) system. By using this native pathway, the antibody specifically binds BBB receptors, translocating into the brain in a controlled and non-damaging manner as a biological “Trojan Horse”, while carrying therapeutic compounds. The potential of this strategy for CNS drug delivery has already been well-validated for two targets, the insulin receptor (IR) and the transferrin receptor (TR). Antibodies towards these two receptors have demonstrated the ability to transport therapeutic drugs across the BBB through RMT, validating the potential of this pathway for therapy and diagnosis of neurological diseases. Yet, IR and TER are not brain specific moieties, being highly expressed in other tissues, and implicated in metabolically crucial cellular functions. As such, antibodies towards these receptors could lead to mistargeting of brain drugs to other sites thus resulting in unwanted side effects and creating safety risks. Moreover, the majority of antibodies developed are IgGs, a class of large molecules which limits their brain accessibility and translocation, having systematically failed to attain sufficient concentrations in the brain side and hampering their therapeutic potential. In addition, the IgG uptake by widely expressed Fc receptors, along with their prolonged half-lives, further contributes for its non-specific accumulation that may result in systemic secondary effects. Therefore, there is an urgent need for identification of more selective BBB targets or improved antibodies that can enhance the uptake of therapeutic molecules into the brain with minimized non-specific accumulation. For targeting and drug delivery functions, the only components of the IgG molecule that are necessary are the antibody variable binding domains, the VH or VL. Their small size enables to reach epitopes inaccessible to conventional IgGs, which, along with the possibility of controlled drug conjugation engineering, paves the way to new drug delivery strategies to successfully transpose the BBB. Moreover, single-domain antibodies (sdAbs) are highly stable moieties, present low immunogenicity, decreased manufacturing cost and their structure allows flexible attachment to neuropharmaceuticals or nanoparticles containing biologically active compounds, making them quite attractive drug delivery vectors.

More recently, a detailed transcriptomic and proteomic analysis of mouse brain endothelial cells allowed the identification of three robust BBB receptors for drug delivery, with translation into the human setting, namely, basigin, glutamate receptor 1 and CD98 heavy chain (CD98hc). Antibodies developed towards CD98hc presented improved brain targeting and drug delivery properties when compared with the IR and TfR.

Nevertheless, these BBB-targets were identified by in vitro selection studies that do not fully mimic the in vivo environment of the BBB and possibly compromise the expression levels and conformation of its native receptors. Indeed, if we look into the in vivo BBB characteristics, it is known that BBB endothelial cells get stimulated by their surrounding cells and intraluminal blood flow. This regulates the expression of specific receptors at the cell surface in a polarized fashion contributing to the complexity of the BBB. For these reasons, screening for highly selective BBB transmigrating antibodies should preferably be performed in vivo.

Therefore, in order to solve these prior art problems, the present invention proposes a new approach that involves the whole cell in vivo immunization in rabbits followed by an in vivo phage display selection in a murine model aiming to develop potent BBB transmigrating nano-antibody scaffolds.

To achieve this, a rabbit derived immunized sdAb library towards brain endothelial cells receptors was constructed and brain specific nano-antibodies were recovered in an in vivo phage display assay.

With this combinatorial approach we successfully identified a panel of novel BBB crossing sdAbs that can specifically target and reach the brain. In order to evaluate the potential of our selected sdAbs for CNS drug delivery, the most promising lead antibody was engineered at liposomes surface encapsulating a model drug that has been demonstrated not to cross the BBB, the pan-histone deacetylase inhibitor (HDACi) panobinostat (PAN), and its BEB transmigrating properties and anti-tumour activity were evaluated in a dual functional in vitro BEB-glioblastoma model.

### Definitions and Abbreviations

aa Amino acids
ABTS 2,2′-azino-bis(3-ethylbenzothiazoline-6-sulphonic acid)
ADCC Antibody-Dependent Cell-mediated Cytotoxicity
Amp Ampicillin
Anti-HA-HRP Anti-Hemagglutinin-Horseradish Peroxidase
BM Bone Marrow
BSA Bovine Serum Albumin
C Carboxylic terminal
CDC Complement-Dependent Cytotoxicity
CDNA complementary DNA
CDR Complementary Determining Region
CH Constant domain of heavy-chain
cNHL Canine non-Hodgkin Lymphoma
CL Constant domain of light-chain
DMEM Dulbecco's Modified Eagle's Medium
DNA Deoxyribonucleic Acid
E. coli Escherichia coli
ELISA Enzyme-Linked Immunosorbent Assay
Fab Fragment antigen binding
Fc Constant Fragment
FDA Food and Drug Administration
LB Luria-Bertani Broth
mAbs Monoclonal antibodies
N Amino Terminal
NHL non-Hodgkin Lymphoma
NK cells Natural Killer
OD Optical density
PBS Phosphate Buffered Saline
PCR Polymerase Chain Reaction
PEG-8000 Polyethylene glycol
pT7-PL pT7-peptide leader
RNA Ribonucleic Acid
RPM Rotations per minute
RPMI Roswell Park Memorial Institute Medium
RT Room temperature (20 to 25° C.)
SB Super Broth medium
scFv Single-chain variable fragment
sdAb Single Domain antibody
SOC Super Optimal Broth
SP Spleen
UV Ultraviolet
VH Variable domain of heavy-chain
VL Variable domain of light chain

### DESCRIPTION OF THE FIGURES

FIG. 1—Characterization of rabbit immune response.

Rabbit immunization: three female New Zealand white rabbits were immunized for 4 months with 1×10⁷ of cNHL primary cells from our biobank. cNHL primary cells from patients diagnosed with DLBCL were selected. Five days after the final boost, rabbits were sacrificed, and spleen and bone marrow were harvested for total RNA isolation and cDNA synthesis. Elisa Serum titration 5×10⁴ cells were incubated with serial dilutions of rabbit serum (from 1/1000 to 1/32000).

All three rabbit samples presented a high response against cNHL primary cells and CLBL-1 cell line.

FIG. 2 (2A, 2B)—FACS analysis confirmed the results obtained by ELISA.

FIG. 3—Protein profile analysis of rabbit serum by immunoblotting of CLBL-1, cNHL primary cells and PBMC from healthy dogs. Results revealed potential tumour-specific epitopes recognized on CLBL-1 and cNHL primary extracts. Representative blots are shown.

FIG. 4—In vitro and In vivo phage display selection. To select the best antibodies for non-Hodgkin Lymphoma (NHL) targeting, sdAb immune library previously constructed with a diversity of 10¹¹⁻¹² was used for an in vitro whole cell and in vivo phage display in a cNHL xenograft murine model.

FIG. 4A—First, a whole cell phage display was performed. The number of washes was increased over the selection to improve the stringency to recover the phage clones with greater affinity and specificity. Two different elution methods were performed to recover binder and internalized antibodies.

FIG. 4B—Following the in vitro selection, a final in vivo phage display was performed in a murine xenograft cNHL model. Briefly, the phages output from the 3^{rd} in vitro panning were recovered, reamplified and tailed vein injected in a xenograft murine cNHL model. After 60 minutes, the mice were euthanized, and the phages recovered from the tumour.

FIG. 5. The selection results in a lower number of phages in the output titters, indicating a high enrichment towards the phages to target cNHL.

FIG. 5A. Results obtained for VH library screening.

FIG. 5B. Results obtained for VL library screening.

FIG. 6. Screening for NHL targeting

FIG. 6A—In order to determine the best lead candidates, around 200 clones were tested in ELISA assays. Three parameters were evaluated: binding, expression and unspecific bindings. The ones that revealed a stronger signal against NHL cell extracts were selected, and

FIG. 6B—The selected 43 best clones were analysed by sanger sequencing.

FIG. 7—To characterize in more detail the enriched sequences during the in vivo phage display next generation sequencing was performed. Two samples were sequenced: biopanning from cNHL and hNHL tumour models and initial immune library.

FIG. 8. Binding and internalization characterization of C5 V_{L} sdAb. C5 binding and internalization properties against CLBL-1 cells were evaluated by cytometry and immunofluorescence. A) For flow cytometry analysis, 1×10⁶ of CLBL-1 cells were incubated with Live/Dead reagents for 30 minutes. Then, 3 μM of C5 were incubated with the cells for different timepoints at 37° C. After, cells were washed, fixed, permeabilized, and incubated for 30 min with anti-HA antibody, washed twice and incubated with anti-rat Alexa Fluor-488. C5 has demonstrated to bind to CLBL-1 cells. B) By contrast, no binding of C5 was detected with Jurkat cells. C) To confirm the binding of C5 to the CLBL-1 verified by flow cytometry, we evaluated its distribution on the cells by immunofluorescence assay. It is possible to observe a high density of Alexa Fluor-488 labelled C5 labelled in the perinuclear region. D) In contrast, there is no detectable fluorescence in the control image, nor in the presence of the Jurkat cells. Representative microphotographs with C5 (green) and DAPI stained-nuclei (blue) are shown.

FIG. 9. Biodistribution profile of ^{99Tm} Tc—C5. To evaluate the tumour al uptake and pharmacokinetic profile of the C5 V_{L} sdAb, a biodistribution assay was performed on a xenograft model of cNHL at two different time points (15 min and 3 h). C5 was radiolabelled with ^{99m}Tc(CO)₃(H₂O)₃ and intravenously injected in the tail vein of a xenograft mice model of cNHL. Mice were sacrificed at 15 min and 3 h and the radioactivity of each organ was measured. The activity in each organ was calculated and expressed as a percentage of injected radioactivity dose per gram of organ or tissue (% ID/g). A) The results show that the tumour al uptake was around 1.5±0.5% ID/g at 15 min, diminishing to 1.5% at 3 h after injection. A fast elimination in the major organs was noticed, with exception of the liver and spleen, C) The results were also confirmed by western blot analysis. Representative blots are shown.

FIG. 10—Bioconjugation of C5-DAB-SN-38. Conjugate C5-DAB-SN-38 obtained from C5 cysteine modification at PBS buffer at pH 7.4 (10 UM) at room temperature. Top right: ribbon representation of C5 sdAb's predicted 3D structure. The CDR domains have been highlighted in purple (CDR1), green (CDR2) and blue (CDR3). The side chain atoms are shown in stick representation and coloured in yellow. The van der Waals surface is depicted in transparent colouring. The surfaces of CDR3 and Cys have been highlighted. CDR and amino acid numbering were done according to Kabat et al. Bottom right: A) High-resolution mass spectra of conjugate C5-DAB-SN-38. B) Deconvoluted high-resolution mass spectra of C5 (12879.2 Da). C) Deconvoluted high-resolution mass spectra of conjugate C5-DAB-SN-38 (13651.5 Da).

FIG. 11—Cytotoxic effects of C5-DAB-SN-38. To determine the effect of C5-DAB-SN-38 on CLBL-1 and Jurkat cells, a cell viability assay was performed using the WST-1 reagent. 6×10⁴ cells were seeded and treated with increasing amounts of compound. After 48 h treatment, cell viability was assessed. A) C5-DAB-SN-38 demonstrated a dose-dependent toxicity effect on cNHL cells. B) On the other hand, ADC had no effect on Jurkat cells, proving the specificity of the ADC. C5 was used as control. Best-fit EC50 values of each formulation were calculated using GraphPad Prism software (version 9.2.0, San Diego, CA, USA) using the log (inhibitor) vs response (variable slope) function. D) Effects of C5-DAB-SN-38 on DNA TopoI activity. To evaluate the effects of C5-DAB-SN-38 on DNA Topo I, we evaluated its activity using the Human Topoisomerase I Assay Kit. After incubation of C5-DAB-SN-38 with 1× reaction buffer and 10 U of Topo I for 1 h at 37° C., the supercoiled DNA was added. To stop the reaction, stop loading buffer was added. DNA TopoI activity was visualized by 1% agarose gel electrophoresis attained with ethidium bromide. Relaxed DNA indicated TopoI activity while supercoiled DNA indicated an inhibition. The presence of C5-DAB-SN-38 at higher concentrations inhibits DNA TopoI in a dose dependent manner, empathizing the effect of SN-38, present on the ADC, as a cause of cell death. Relaxed DNA and supercoiled DNA were used as positive/negative controls. C5 and SN-38 were also used as controls.

FIG. 12—Schematic representation of the in vivo screening process to select highly specific BBB-crossing sdAb.

FIG. 13. Analysis of rabbit immunological response, wherein:

FIG. 13A. ELISA serum titration of Rabbit #1 and Rabbit #2 total serum against bEnd.3 endothelial cells. Total rabbit serum was diluted by serial dilution (from 500 to 32,000-fold) and tested in 2×10⁴ cells/well. Results showed a selective and specific immune response against bEnd.3 cells, and

FIG. 13B. Target validation and assessment of potential bEnd.3 receptors recognized by each rabbit serum following immunological boost by WB. Total protein extract was obtained with RIPA buffer from bEnd.3 and 10 μg and 20 μg was loaded in a 11% SDS page acrylamide gel. Following gel transfer and membrane blockage, total rabbit serum 500-fold diluted was added to determine the specificity of the antibodies produced by the rabbit immunization process towards bEnd.3 total protein extract. Results demonstrated that antibodies towards several receptors were developed in both immunized rabbits validating our immunization approach.

FIG. 14. Validation of the rabbit serum BBB crossing features. To confirm that the antibodies obtained from the rabbit immunization process retained the ability to cross the BBB, purified serum from both rabbits were tested in both in vitro and in vivo models, wherein. For the in vitro BEB model 4×10³ bEnd.3 cells were seeded in 24-well plates tissue culture inserts and incubated for 11-14 days in a humidified chamber at 37° C., supplemented with 5% CO₂,

FIG. 14A. To determine the rabbit serum translocation efficiency, 10 μg of purified serum was added to the apex, incubated for 15 and 60 min, recovered from both the apex and the base, and analysed by WB. Rabbit serum was detected at the base of the monocellular model in both time points validating the translocation ability of the rabbit IgGs,

To confirm the ability to reach the brain in an in vivo model, purified serums (250 μg) were intravenously injected into the tail vein and after 2 and 60 min the mice were sacrificed, and the brain extracted

FIG. 14B. Rabbit immunoglobulins were recovered from the mice blood and homogenized brains by IP with protein A beads and analysed by WB with goat anti-rabbit-HRP IgG at 1:1000. The detection of the rabbit serum in the brain extract validated the ability of rabbit derived antibodies to reach the mice brains and further confirmed the anticipated species cross-reaction.

FIG. 15. Selection of sdAbs by in vivo phage display and NGS. In order to elect the most promising sdAbs for brain targeting and BBB transposition, the rabbit derived immunized phage displayed library was intravenously injected into the tail vein of CD1 mice. Phages were allowed to circulate for 2 and 60 min and the mice were perfused, sacrificed and phages extracted from the brain were re-amplified. Three rounds of in vivo biopanning were performed, in which the phages recovered from the brain were reamplified and re-injected into the mice for a new round of selection. For each selection round, quantification of the injected phages (input) and the phages recovered from the brain (output) was determined. Following in vivo selection, the selected phage library was re-amplified and analysed by next-generation sequencing and data analysed by an in-house bioinformatic script. Sequences with more than 20 representatives in at least one time point are presented. The clones selected for in vivo biodistribution, following expression and stability assays, are depicted.

FIG. 16. In vitro selection of the dominant sdAbs clones. With the aim of selecting the most competent clones in transposing the BBB, the clones with higher frequency in NGS analysis and proficient in terms of expression and solubility properties (data not shown) were selected for further characterization in terms of in vitro BEB model translocation efficacy.

FIG. 16A 15 μg of each purified sdAb was added to the apex of the transwell and incubated for 90 min.

FIG. 16B Following incubation period, all volumes were collected from the apex and the base and analysed by WB with anti-HA antibody.

FIG. 17. Translocation of RG3 and FC5 functionalized liposomes in the in vitro BEB model (Lip-RG3 and Lip-FC5). To determine the effect of RG3 functionalization in liposome in vitro BEB translocation, rhodamine loaded, biotinylated liposomes were surface modified with RG3 and added to the apex of the transwell, as described previously, and incubated for 90 min (FIG. 17A), 6 h (FIG. 17B) and 24 h (FIG. 17C). The translocation percentage was determined by measuring the rhodamine fluorescence intensity at the base. Non functionalized liposomes (Lip) and FC5 functionalized liposomes (Lip-FC5) were used as controls. A one-way ANOVA statistical test followed by a Turkey's test was used to compare each liposomal formulations (**** p<0.0001; *** p<0.001; ** p<0.01; * p<0.05; n.s., not statistically significant).

FIG. 18. Activity of liposomal drug delivery in the BEB-Glioblastoma model, wherein an in vitro BEB-GBM model was developed to mimic the complexity of drug delivery to a brain tumour. This model combined a monolayer of bEnd.3, as the BBB barrier, and the LN229 cell line at the base as the glioblastoma tumour.

FIG. 18A. Shows the evaluation of the ability of free PAN to translocate the BEB and its effect on the integrity of the model barrier, PAN was added at increasing concentrations to the apex of the BEB-GBM model and incubated for 24 h. The integrity of the bEnd.3 was measured by determining the translocation of the FD40 fluorescent probe while LN229 viability was determined using WST-1 reagent and OD_{450 nm} measurement following 24 h incubation, and

FIG. 18B. PAN encapsulated RG3 and FC5 functionalized liposomes were analysed in terms of BEB translocation and delivery of PAN to a glioblastoma cell line and compared with non-functionalized unloaded liposomes (Control). Briefly, PAN loaded liposomes were added to the apex of the BEB-GBM model and incubated for 24 h. The cytotoxic effect of PAN in LN229 was determined by adding WST-1 reagent. The values were obtained from duplicates of two independent experiments (FIG. 7B1, 7B2).

### DESCRIPTION OF THE INVENTION

The present invention relates to the development of antibody fragments as alternative targeting agents for drug delivery systems, such as single domain antibodies (sdAbs).

For this purpose, antibody-drug conjugates (ADCs) were developed with high selectivity and efficiency to be used advantageously in cancer therapy and in central nervous system (CNS) pathologies.

In one aspect, the present invention relates to ADC molecules developed for therapy, namely for cancer therapy, comprising rabbit derived sdAbs and a potent cytotoxic payload conjugated with the free exposed cysteine at position 80 of the V_{L} framework.

Currently, sdAbs are the smallest functional antibody fragments, only consisting of a VH or VL unit. These small size scaffolds of about 15 kDa possess higher tumour penetration and accessibility to targets not easily reached by large size conventional mAbs. Moreover, their faster clearance rate compared to the intact IgG, may be advantageous in cases where the risk of toxicity in healthy tissues increases with prolonged exposure.

In addition to their reduced size, sdAbs also present higher stability, solubility, lower immunogenicity and lower manufacturing costs, as they can be expressed in bacterial systems. Importantly, rabbit-derived V_{L} sdAbs have, in their natural framework region, a free exposed cysteine at position 80 that can be explored to selectively conjugate a chemical payload, without requiring further genetic engineering manipulation.

In result of the above, the ADC molecules of the present invention, comprise a potent payload adequate to kill target cancer cells, SN38 small molecule conjugated with a free cysteine.

It is also disclosed the process to obtain such ADCs and their use as medicaments. The process of obtaining such molecules includes the selective conjugation of a free cysteine present in rabbit derived sdAbs with a chemical payload, without requiring further genetic engineering manipulation.

In another aspect, the present invention relates to molecules specifically developed to central nervous system (CNS) therapy, that are able to cross the blood-brain barrier (BBB). These drugs also include rabbit derived single-domain antibodies (sdAb) specifically targeting BBB endothelial cell receptors.

It is also disclosed the process to obtain these molecules by using a construct of rabbit derived single-domain antibody (sdAb) library conjugated at the surface of liposomes encapsulated with a suitable drug enabled an efficient BBB translocation and presented a potent antitumour al activity.

### I. Description of the Process for Producing an Anti-Tumour ADC According to the Preferred Embodiment of the Invention

According to the preferred embodiment of the invention it is provided a drug delivery system VL-DAB-SN38 for using in the treatment or tumour cell-therapies, comprising a VL chain, engineered to exhibit a single free exposed cysteine at position 80 of the V_{L} framework, was modified with the DAB-SN38, a molecule containing the cytotoxic drug SN38 and a maleimide, bonded through a diazaborine bioconjugation linker.

The processes for obtaining the VL-DAB-SN38 drug delivery system can be described as comprising the following main steps:
a) Providing lymph node primary cells derived from a canine multicentric lymphoma biobank,
b) Rabbit immunization with 1×10⁷ of lymph node primary cells derived from a canine multicentric lymphoma biobank
c) Isolation of RNA and cDNA from samples of spleen and bone marrow,
d) Using a constructed single domain antibody (sdAb) targeting cNHL,
e) Using a rabbit sdAbs in the format of a V_{L} light chain variable region with the free exposed cysteine at position 80 of the V_{L} framework,
f) Binding of VLs to cNHL cells with incubation of anti-HA FITC antibody,
g) Bioconjugation of VL with DAB-SN38 by adding a solution of DAB-SN38 to a solution of VL with TCEP.

In a preferred embodiment more than 200 clones present anti-tumour properties.

In a more preferred embodiment, 43 clones presented a strong signal against non-Hodgkin Lymphoma (NHL) cells (SEQ. ID No. 1 to SEQ. ID No. 43, in the Sequence listing).

Amongst them 6 clones presented the best results: A12, C8, C5, E1, E2, B12, with the following sequences:
SEQ. ID. 1 (Clone A12)
SEQ. ID. 2 (Clone C8)
SEQ. ID. 3 (Clone C5)
SEQ. ID. 4 (Clone E1)
SEQ. ID. 5 (Clone E2)
SEQ. ID. 6 (Clone B12)
   1. Generation and Screening of High-Tittered Antisera Against cNHL

To develop highly specific cNHL sdAbs, first, a highly specific sera against cHHL antigens is produced by immunizing selected rabbits with lymph node primary cells derived from a canine multicentric lymphoma from the Faculty of Veterinary Medicine, University of Lisbon.

The immunization can be monitored by antibody titters, and specificity by ELISA and FACS.

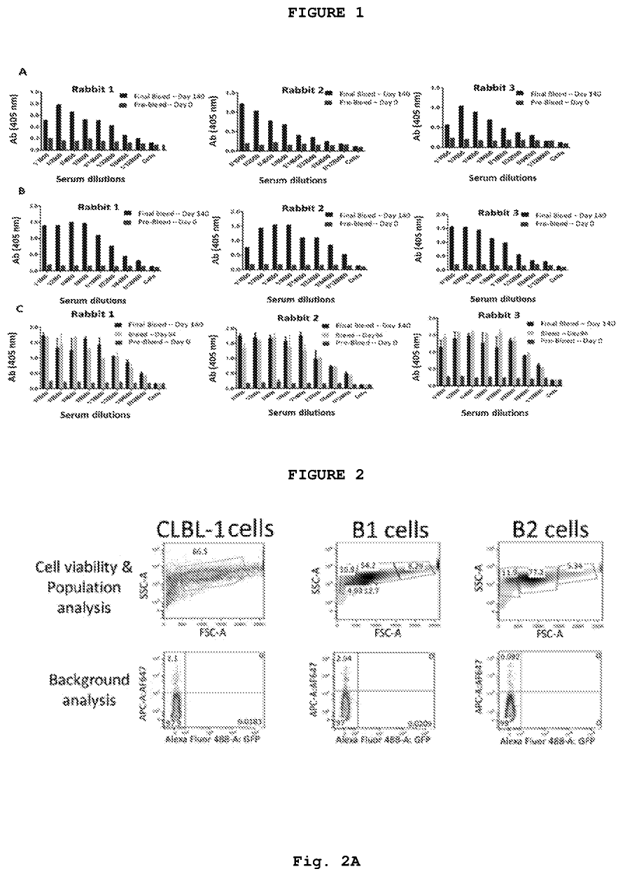

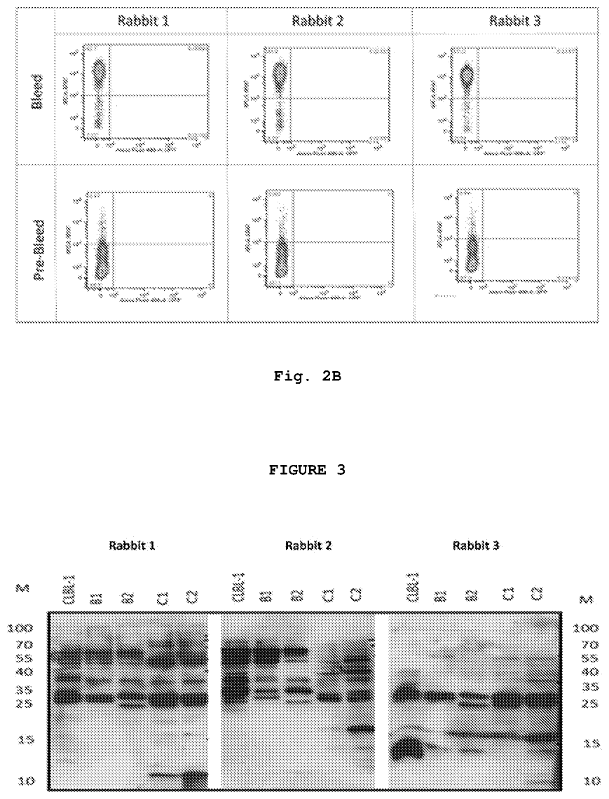

As shown in FIG. 1, cell ELISA assays showed that all three rabbits final bleed serum presented a highly specific and selective response against our biobank cNHL primary cells and CLBL-1 cell line (FIG. 1), a canine B-cell lymphoma cell line. Furthermore, these data demonstrated that immunizations resulted in a strong immune response with a high serum titter (1:60.000), contrarily to pre-bleed serum. FACS analysis confirmed these results (FIG. 2). To evaluate the protein profile recognized by rabbit serum, immunoblotting was performed. As shown in FIG. 3, protein profile recognized on cNHL primary and CLBL-1 extracts were similar. On the other hand, various differences were detected among the receptors recognized on PBMC. The results obtained revealed potential tumour-specific epitopes detected by rabbit antibodies present on cNHL cells, unveiling a therapeutic potential of these antibodies.

### 2. Construction of the Immunized Library and Phage Display Selection

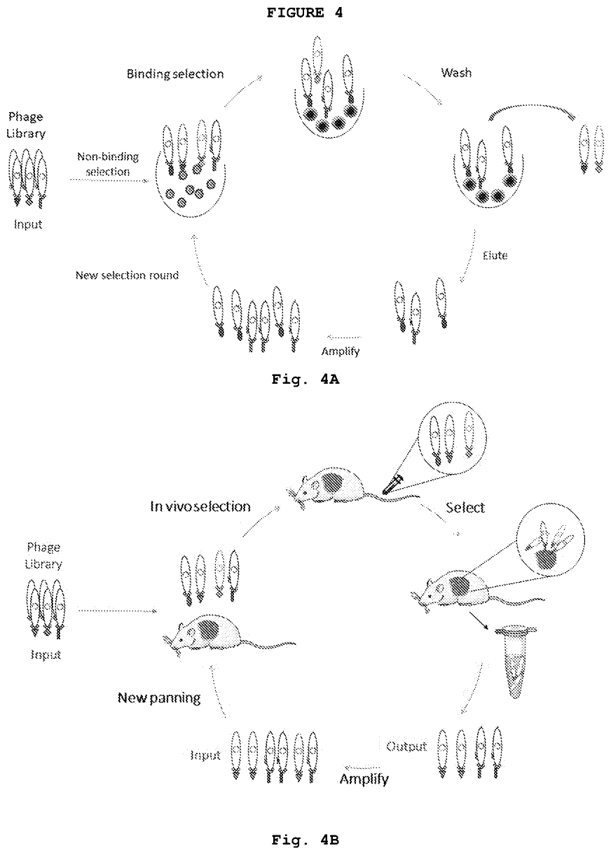

To select the most promising antibodies for NHL targeting, a sdAb library is provided or constructed by amplification of the V_{L}, recovered from bone marrow and spleen of selected immunized rabbits with both cNHL primary cells presenting Diffuse Large B Cell Lymphoma (DLBCL) from the biobank. The V_{L} sdAbs regions are then cloned in the pComb3X phagemid vector originating a phage displayed library with a diversity of 10¹¹⁻¹² that is used for an in vitro and in vivo phage display selection in a murine model (FIG. 4).

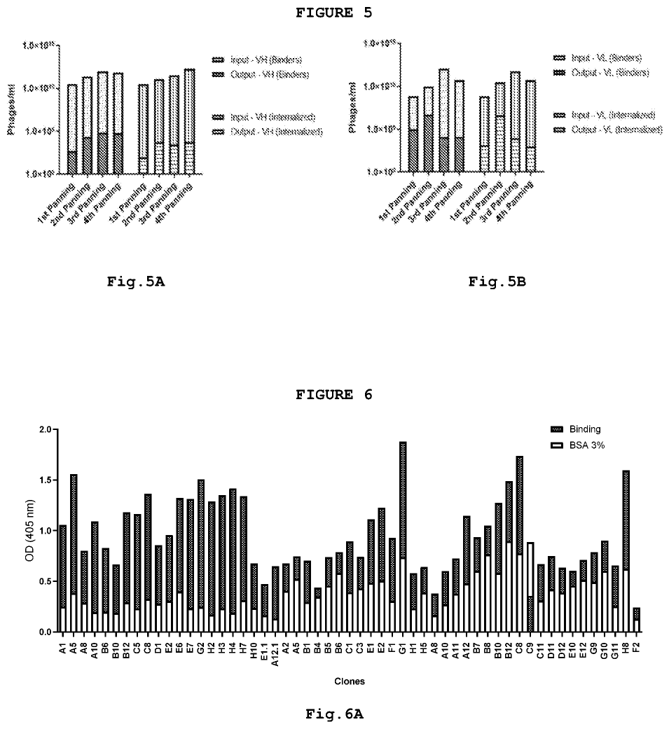

First, a whole cell phage display is performed to select the most promising sdAbs targeting cNHL epitopes (FIG. 4A). This cell phage display screening protocol is based on the previously Carlos Barbas and our studies (Barbas III, C. F., Burton, D. R., Scott, J. K. & Silverman, G. J. Phage Display: A Laboratory Manual. (Cold Spring Harbor Laboratory Press, 2001) and Dias, J. N. R. et al. Characterization of the canine CD20 as a therapeutic target for comparative passive immunotherapy. Sci Rep 12, 2678 (2022).), in which it is reported a novel whole-cell selection protocol designed to select mAbs recognizing surface epitopes, that potentially internalize. Over the course of selection, stringency can be improved by increasing the number of washes in order to collect the phage clones with greater target affinity or specificity. Different elution methods can be performed to select for adequate binders and internalized antibodies. As shown in FIG. 5, the three in vitro phage display pannings resulted in a lower number of phages in the output titters compared to the input titters (˜10¹¹⁻¹² to ˜10³⁻⁴ pfu). Furthermore, the biopannings profile indicates that the in vitro phage display successfully led to the enrichment for highly specific cNHL sdAbs. Following the in vitro phage display selection, a final in vivo phage display panning was performed in a murine xenograft NHL model (FIG. 4B). The phage displayed output recovered from the 3^{rd} in vitro panning was recovered, reamplified and tailed vein injected in a xenograft murine cNHL model previously described by Dias et al. in Dias, J. N. R. et al. Establishment of a bioluminescent canine B-cell lymphoma xenograft model for monitoring tumour progression and treatment response in preclinical studies. PLOS ONE 13, e0208147 (2018). At 60 min post injection the mice were euthanized, the tumours removed, and phages recovered to retrieve Cnhl specific sdAbs. By allowing the enriched antibody panel to circulate in the mouse and collecting sdAbs that targeted the xenograft tumour, we expected to select antibodies that met the defined biological effect, confirming the in vivo availability of the epitopes. At the end of this in vivo panning, the phages recovered presented a diversity of 10⁶ of V_{L} binders and 10⁴ of V_{L} internalizers (phages/Ml), which reflected a high enrichment towards the phages that present an increased ability to target cNHL.

### 3. Screening for NHL Targeting

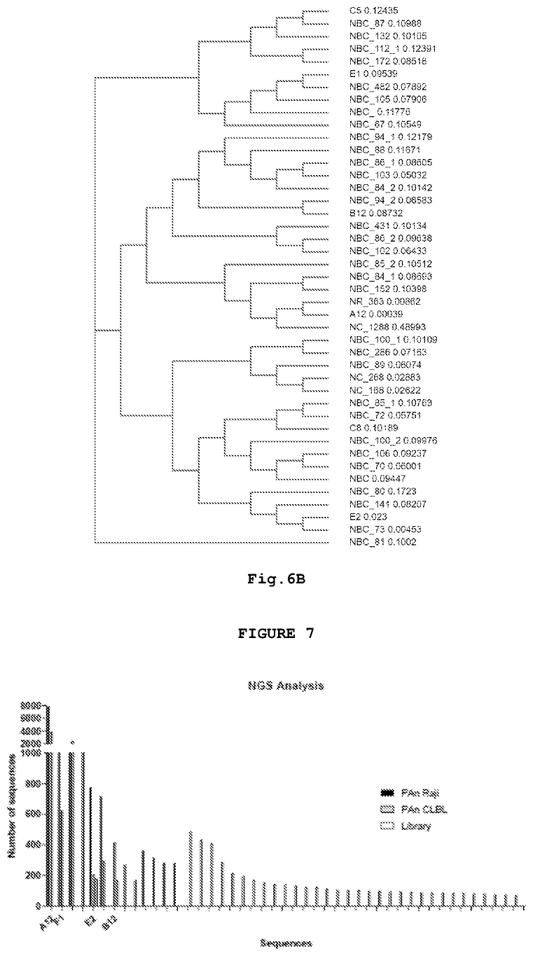

After the phage display selection, in order to express and select the best anti-cNHL V_{L} sdAbs, phagemid DNA derived from the in vivo output selection was cloned into a PT7-PL vector and transformed into an E. coli strain BL21. Then, individual clones were auto induced, and the supernatant tested in ELISA assays against CLBL-1 and Jurkat cell extracts. To select the best lead candidates, three parameters were evaluated: binding against cNHL, expression yields, and unspecific binding. Around 200 clones were screened and the V_{L} sdAbs that revealed a stronger signal against CLBL-1 were selected (FIG. 6A). After selection, to characterize the selected clones, the 43 best lead candidates were analysed by sanger sequencing (FIG. 6B). To get further insights of the enriched sequences during the in vivo phage display selection, we performed next generation sequencing (NGS) of the biopanning repertoires. NGS allows massive sequence analysis of the panning population, enabling a genomic assessment of the library diversity and frequency of each clone. Two samples were sequenced: biopanning from the CLBL-1 tumour models and initial immune V_{L} sdAb cNHL library (FIG. 7). Upon bioinformatic analysis of the NGS data, we were able to determine the diversity of each sample and identify the most represented clones. A total of 34880 sequences were obtained for the biopanning from the CLBL-1 tumours. The NHL library obtained a total of 48864 sequences. The number of singletons, consisting of the sequences with a single occurrence, varied between samples, ranging from 76.8% in the library, and 33.3% in the in vivo biopanning from the CLBL-1. Through the comparison of the sequence prevalence between the biopannings and the library, we were able to verify that the number of occurrences in each sequence was higher in the biopanning clones. These results evidenced the specificity of the phage display selection, diminishing the high diversity of clones that were present in the library. By comparing ELISA data, sanger sequencing and NGS analysis, the six best clones (A12, C8, C5, E1, E2, and B12) were produced and purified. Due to its binding characteristics and production properties, C5 sdAb clone was chosen to be thoroughly characterized and used in the development of the proposed ADC.

### 4. Characterization of Cell Binding by FACS

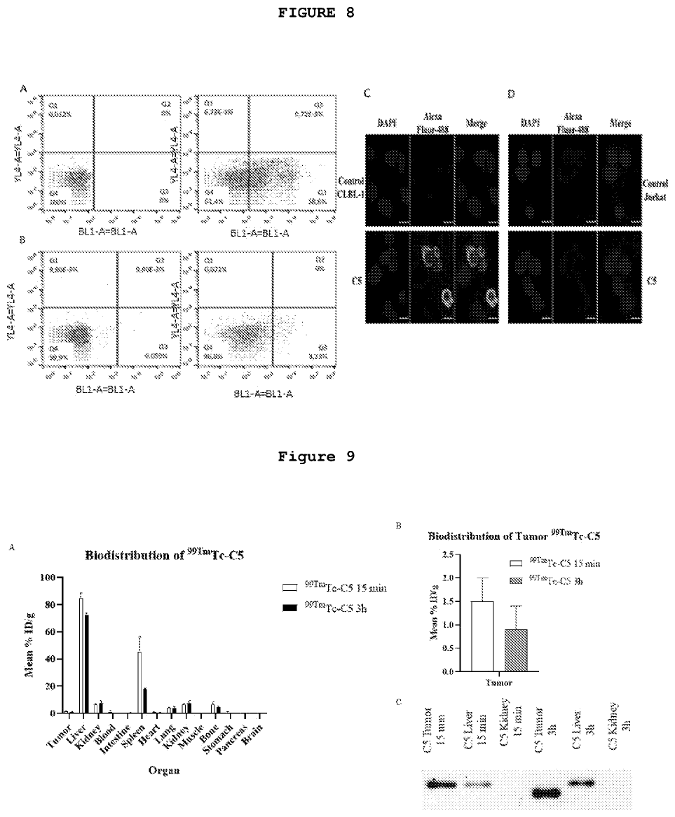

To evaluate the binding of the VL C5 to the CLBL-1 cells, a FACS analysis was performed. VL was incubated at different timepoints with cHNL cells. As shown in FIG. 8 (panel A and B), VL has been shown to bind to the cells, increasing its interaction with time. Controls demonstrated no interaction with the antibody.

### 5. Assessment of Cell Binding by Immunofluorescence

To follow up the binding of the VL C5 to the CLBL-1 cells by FACS analysis, we further evaluate the distribution of the antibody on the cells using an immunofluorescence assay. As shown in FIG. 8 (panel C and D), is possible to observe a high density of VL C5 with Alexa Fluor-488 in the perinuclear region. Conversely, there is no detectable fluorescence in the control image. Thus, these images confirmed the V_{L} binding to the CLBL-1 cells and its internalization into the cytoplasm. This internalization feature was essential to develop an efficacious ADC.

6. Biodistribution Studies of C5 V_{L} sdAb

To evaluate the tumour al uptake and the pharmacokinetics profile of the selected C5 V_{L} sdAb, a biodistribution assay was conducted on a xenograft model of cNHL at two different time point (15 min and 3 h). For that purpose, C5 was radiolabelled with ^{99m}Tc and intravenously injected into mice tail as described in the material and methods section. The obtained biodistribution profile of the labelled ^{99m}Tc—C5 V_{L} sdAb, expressed as % ID/g, is presented in FIG. 9. The data obtained showed that the tumour uptake was around 1.5% ID/g at 15 min, decreasing to 1% at 3 h after injection. In addition, except the liver and spleen, the biodistribution data revealed a fast elimination from blood and major organs, with low levels of activity. These results were also confirmed by western blot analysis confirming the presence of the C5 V_{L} sdAb (FIG. 9) in CLBL-1 xenograft tumours.

### 7. Bioconjugation of VL-DAB-SN38

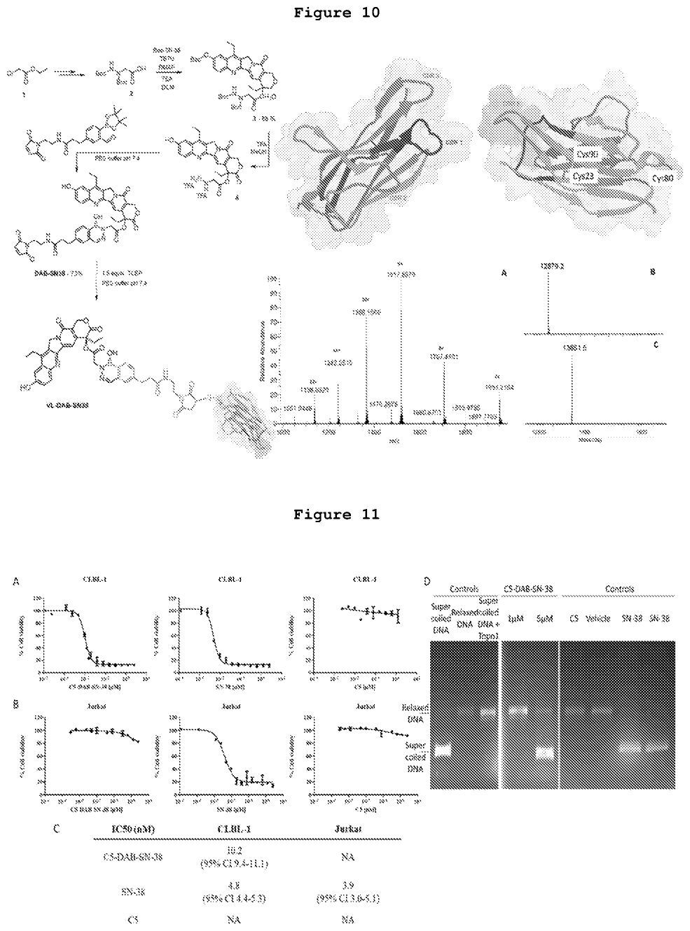

Aiming to develop the ADC by exploring the free cysteine at position 80, the predicted tridimensional structure of the C5 V_{L} sdAb was determined. The obtained structural model revealed a traditional immunoglobulin domain fold composed of eight antiparallel β-strands arranged into two β-sheets, which are connected by a single disulphide bond formed between Cys23 and Cys90, forming a β-sandwich (FIG. 10). The three CDR motifs are easily identifiable, with CDR3 having the larger area of exposed surface. Importantly, the structure shows the presence of a third cysteine (Cys80) on its surface with a free sulfhydryl group, which is normally involved in a rabbit-unique interdomain disulphide bond with another cysteine on the CL domain. As predicted, by isolating the V_{L} domain, Cys80 becomes exposed at the protein surface and its sulfhydryl group becomes free to be used in cysteine-based conjugation strategies. As such, C5 sdAb single free cysteine was modified with DAB-SN-38, a molecule containing the cytotoxic drug SN-38 and a maleimide group, connected by a ROS-responsive diazaborine linker (FIG. 10). The C5 V_{L} sdAb was successfully converted in the homogenous targeting drug conjugate, C5-DAB-SN-38.

8. Evaluation of Cytotoxic Activity on cNHL Cells

After the conjugation of the VL-DAB-SN-38, its in vitro activity was evaluated. For that, a cell viability assay on CLBL-1 and Jurkat cells was conducted using WST-1 reagent. As shown in FIG. 11A-c, ADC demonstrated dose-dependent effects on toxicity CNHL cells proliferation. By contrast, VL-DAB-SN38 had no effect in the proliferation of Jurkat cells, a leukemic T-cell line, reinforcing the specificity of the constructed ADC. Furthermore, cell viability in the presence of VL was also evaluated, leading to the conclusion that the antibody alone does not affect cell proliferation in both cell lines.

### 9. Evaluation of Topo I Activity

To determine the effects of VL-DAB-SN-38 treatment on DNA Topo I, we evaluated its activity. DNA TopoI activity is inhibited in the presence of SN-38, inhibiting the conversion of supercoiled DNA into a relaxed DNA form. As shown in FIG. 11D, the presence of VL-DAB-SN-38 at higher concentration inhibits DNA Topo I as we can see by the presence of the supercoiled DNA form. Moreover, VL-DAB-SN-38 seems to inhibit the enzyme in a dose-dependent manner. On the other hand, VL portion was not able to alter enzyme activity, reinforcing the effect of SN-38, present on the ADC, as the cause of cell death. These findings support that cell death has been prompted by DNA Topo I inhibition driven by SN-38 present on the ADC.

### In Conclusion

The addition of Rituximab, a mAb targeting the CD20 receptor, has led to a paradigm shift in the treatment of haematological malignancies in both first line and refractory/relapse settings. However, ensuing experience using alternative unconjugated mAbs, which target other tumour cell receptors, has demonstrated that the clinical efficacy attained with these mAbs is frequently limited.

The development of ADCs showed a most promising strategy to optimize mAb-based therapies efficacy taking advantage of the selectivity of antibody-antigen binding to deliver potent cytotoxic molecules directly and specifically to cancer cells.

This emerging class of therapeutics has proven clinical efficacy in many types of haematological cancers.

Considering the high cytotoxic potency of the payloads used in ADCs, even lower levels of systemic exposures can result in significant toxicity. Within this context, the present invention aims to develop a new generation of highly selective and specific ADC for cancer treatment comprising rabbit derived sdAbs and the payload conjugation on the free exposed cysteine at position 80 of the V_{L} framework. It is herein demonstrated an improved cytotoxicity activity of the ADC in cancer models, confirming the potential of these molecules.

The drug delivery systems herein disclosed are based on rabbit derived antibodies for canine lymphoma as an animal model of human NHL, which prove to be a realistic opportunity for rapid and clinically relevant translation of novel immunotherapies.

NHL is one of the most common types of cancer and one of the fastest growing in incidence in humans.

Owing to remarkable similarities with its human counterpart, the canine lymphoma model has been proposed as a powerful framework for rapid and clinically relevant translation of novel immunotherapies. Hence, herein disclosed is a novel class of rabbit deriving sdAb-based ADC for the treatment of cNHL, that serves as an animal model for hNHL.

Due to their unique B-cell ontogeny, rabbit antibody derived libraries present a highly distinctive and diverse antibody repertoire, rich in in vivo pruned binders of high diversity, specificity and affinity. Importantly, rabbits are evolutionarily distant from mice and rats, so epitopes that are not immunogenic in rodents can be recognized by rabbit mAbs, increasing the targetable epitopes and facilitating the generation of mAbs that cross react with other species—a key aspect for clinical translational. Rabbit immunizations with intact B-cell canine lymphoma primary cells resulted in a specific and selective high-tittered antiserum against NHL epitopes in all animals.

The strong and specific response generated allowed the construction of an antibody library highly diverse and representative. Thereafter, a strategy of in vitro whole-cell phage display, followed by an in vivo phage display in an NHL xenograft model, was used to select the best sdAbs targeting antigens in their natural environment.

This methodology allowed to select both phages that bind and internalize to the tumours' surface. One of the particularities of this final in vivo selection is the naturally occurring negative selection. This enables the reduction of the off-target tissue and protein interactions by eliminating non-specific ligands, enriching the recovery of target-specific ligands.

To the best of our knowledge, this is the first time that an in vivo selection has been applied in the selection of sdAbs against lymphoma malignancies. Overall, data presented herein reinforces in vivo phage display selection as a powerful technology that has the potential to expand the repertoire of targetable tumour receptors, while simultaneously confirming the availability of the epitope in vivo and generating new antibodies for targeting.

Hereupon, an ELISA screening allowed the selection of the best sdAbs candidates targeting cNHL, concerning binding activity and expression. In parallel, NGS analysis was performed to compare the recovered in vivo bioppaning with the library and the clones selected. The NGS analysis enabled us to demonstrate the specificity attained by the phage display in comparison with the initial library. Furthermore, through NGS it was possible to validate the performed ELISA screenings, once the most prevalent sequence on the biopanning was identified as one of the best 6 clones selected by ELISA.

Based on its binding and expression properties, C5I revealed to be the most promising sdAb targeting NHL and was selected for further characterization by FACS and immunofluorescence. Characterization studies allowed us to verify the interaction of the C5 with the CLBL-1 cells and its posterior internalization. The specificity of the interaction of the antibody with the antigen and subsequent internalization of the complex are essential to the success of the ADC as well as to diminish the off-target effects. Thus, the internalization combined with the unique characteristics of the sdAbs, such as their reduced size, makes them great candidates to attach to other molecules, without affecting their activity or stability.

The choice of a potent payload is critical to optimize the already evidenced benefits of our antibody. SN-38 is an active metabolite of the irinotecan, derived from the camptothecin. This molecule interacts with Topoisomerase I (TopoI) that plays a fundamental role during transcription and replication. SN-38 acts as a Topo I inhibitor through binding and stabilization of the TopoI-DNA cleavage complexes, leading to DNA damage and then apoptosis when transcription and replication occurs. There is evidence that the susceptibility of the cells to topoisomerase poisons depends on the amounts of the enzyme inside the cell. It is known that cancer cells express higher yields of Topo I, making its expression 14-16 times higher than in normal cells. This augmented yield of the enzyme is particularly observed in certain types of cancer, including NHL.

Considering the potential of this payload, C5I was bioconjugated to SN-38 using the DAB linker, generating a novel ADC-VL-DAB-SN-38. The obtained data showed that VL-DAB-SN38 promotes cell death on canine lymphoma cells.

In addition, results demonstrated that VL-DAB-SN-38 cytotoxicity on the canine lymphoma is associated with DNA TopoI inhibition. Noteworthy, these results showed that the generated ADCs were stable and presented a high cytotoxic activity against canine diffuse large B-cell lymphoma under the nM range, revealing the potential of these rabbit derived sdAbs as ADC moieties.

### II. Description of the Process for Producing BBB Single Domain Antibodies According to a Second Preferred Embodiment of the Invention

As mentioned above, the present invention also relates to the development of drug delivery systems comprising single domain antibodies (sdAbs).

Thus, in a second aspect of the invention, drug delivery systems are developed for targeting the BBB endothelial cell receptors of the central nervous system (CNS). These systems comprise rabbit derived single-domain antibodies (sdAb) conjugated at the surface of liposomes encapsulated with a suitable drug enabling an efficient blood-brain barrier (BBB) translocation. The respective process of production is also herein disclosed.

For this purpose, the construction of a rabbit derived single-domain antibody (sdAb) library towards BBB endothelial cell receptors is herein disclosed. The sdAb antibody library can be used in an in vivo phage display screening as a functional selection of novel BBB targeting antibodies. Following three rounds of selections, next generation sequencing analysis, in vitro brain endothelial barrier (BEB) model screenings and in vivo biodistribution studies, five potential sdAbs were identified, three of which reaching >0.6% ID/g in the brain.

To validate the brain drug delivery proof-of-concept, the most promising sdAb, namely RG3 (SEQ. ID n. 44), was conjugated at the surface of liposomes encapsulated with a model drug, the pan-histone deacetylase inhibitor (PAN). The translocation efficiency and activity of the conjugate liposome was determined in a dual functional in vitro BEB-glioblastoma model. The RG3 conjugated PAN liposomes enables an efficient BEB translocation and present a potent antitumour al activity against LN229 glioblastoma cells without influencing BEB integrity.

### 3. Results and Discussion

3.1. Construction of the Immunized sdAb Library and Antibody Validation

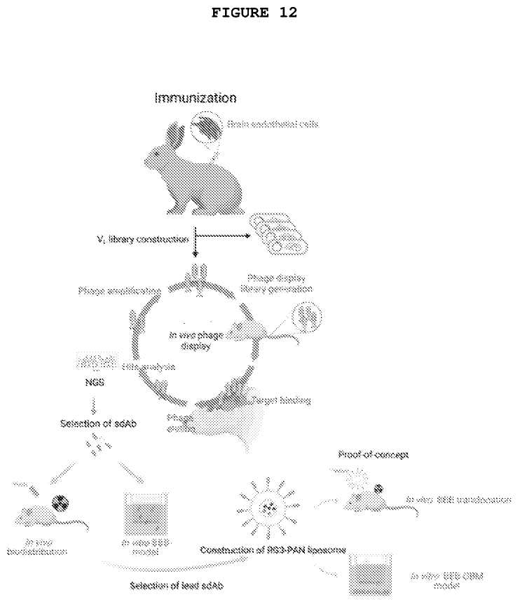

To address the complexity of the BBB, a phenotypic antibody search and identification approach that considers the native conformation of BBB cell surface receptors was developed (FIG. 12).

The nano-antibody sdAb library was constructed by whole-cell immunization of two New Zealand White rabbits with a cell line of mouse brain endothelial cells (bEnd.3). Rabbit antibodies are well known for their ability to produce high affinity and site-specific antibodies, being capable of generating antibodies that recognize similar epitopes from different species. More importantly, contrary to other rodents, rabbits develop highly diverse and strong immune responses particularly against low abundant proteins or hidden epitopes, particularly prevalent in the BBB receptome.

The rational use of the bEnd.3 cell line relates with the subsequent in vivo assays for validation of our immunization strategy.

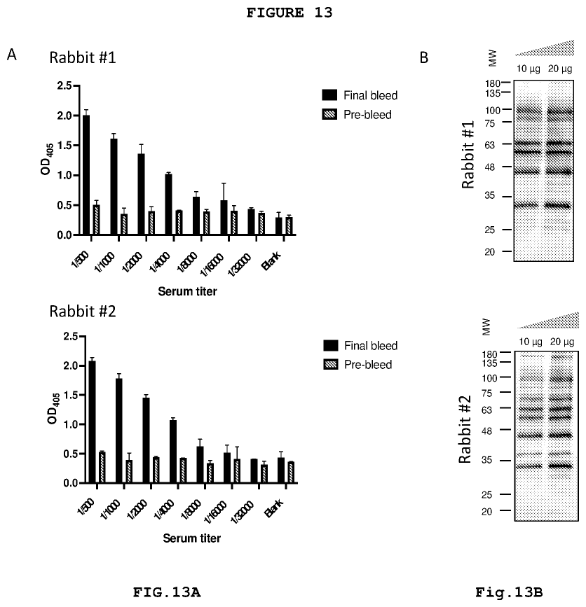

To monitor the rabbit immunological immunization response, bleeds were taken before and after each immunization boosting and evaluated by ELISA (FIG. 13A) and Western Blot (WB) (FIG. 13B) to confirm the serum specificity and titter against bEnd.3 surface receptors. The results obtained for the sera of the two rabbits showed that the immunization process originated a strong and specific immune response against the BEB cellular model receptors, with no antibody recognition of the bEnd.3 receptors in the pre-immunization serum.

Considering that the major goal of the invention is the identification of antibodies capable of translocating the BBB, the ability of the final serum from each rabbit to transpose the BBB is assessed in a well characterized in vitro BEB model composed of a monolayer of brain endothelial cells).

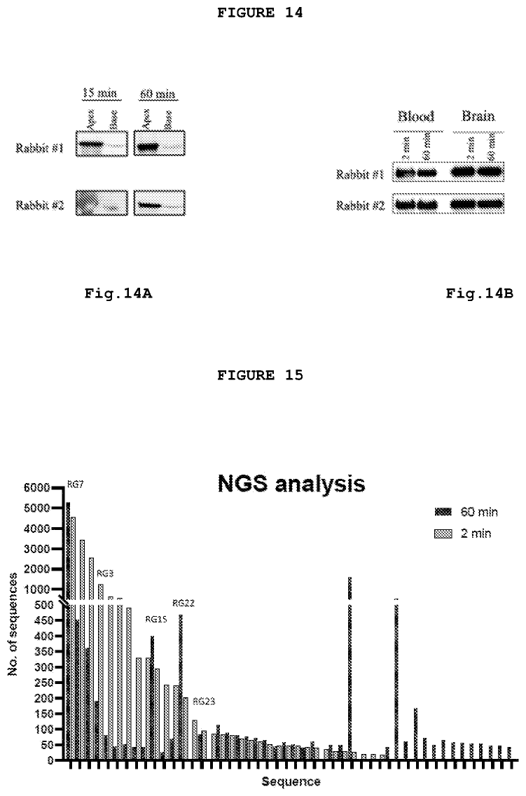

To determine the translocation properties of the rabbit polyclonal sera, purified final serum was added to the apex and incubated for two time points chosen based on previous optimization assays. As shown in FIG. 14A, WB analysis of the rabbit final serum confirmed the presence of antibodies with the ability to translocate into the basal compartment of the monoculture barrier model, with transposition observed at 15 and 60 min.

All in vitro BEB model assays were monitored for cellular integrity following antibody incubation, based on the permeability of the barrier to FD40 probe translocation. Paracellular leakage was negligible in all cases.

Despite its high reproducibility, a major weakness of in vitro BEB models is the decreased complexity in terms of receptor expression when compared to in vivo, which could bias the antibody validation process. As such, to confirm that the rabbit derived antibodies were reaching the brain in a more dynamic model, purified rabbit serum was intravenously injected in CD1 mice (and, following 2 and 60 min post-injection (p.i.), recovered from the blood and brain by immunoprecipitation with protein A. As demonstrated in FIG. 14B, antibodies retrieved from both immunized rabbits were detected in the mice brains, validating the immunization strategy and the presence of antibodies with BBB-crossing features.

3.2. In Vivo Phage Display Selection of BBB-Targeting sdAbs

Following rabbit immunization and validation of the immune response towards brain endothelial cells, a selection of the most proficient antibodies for BBB translocation performed. Aimed at that objective, a sdAb library was constructed by amplification of the antibody light chain variable regions (V_{L}) recovered from the bone marrow and spleen cDNA of the two immunized rabbits. The V_{L} sdAbs regions were then cloned in the pComb3X phagemid vector originating a phage displayed library with a diversity of 1.2×10⁸.

A major advantage of using sdAb is the possibility of generating large libraries that can be used to screen a diverse set of receptors, enabling the discovery of new targets. A powerful technique for selection of antibodies is phage display since the antibodies that are displayed at phage surface can be selected in the intricated milieu of the animal based on desired pharmacokinetic and targeting specificity properties. Moreover, antibodies are identified and tested functionally, and must overcome natural barriers and mechanisms of degradation and thus, the screening for highly specific BBB transmigrating antibodies should preferably be performed in vivo.

Thus, to select a panel of rabbit derived brain targeting sdAbs in a natural context, preserving the in vivo BBB characteristics and its innate intracellular interaction with the surrounding cells and intraluminal blood flow, an in vivo phage display selection was performed FIG. 15A. Briefly, the phage displayed library (input) was injected in the tail vein of CD1 mice. At 2 or 60 min p.i. the mice were perfused, euthanized, the brains were removed, and phages were recovered to retrieve brain specific sdAbs. The time points were selected based on preliminary assays with a naïve library (data not shown) in order to foster the selection of sdAbs that can swiftly target the BBB with limited brain accumulation features. Two subsequent rounds of selective screenings were performed to enrich the BBB targeting sdAbs population (FIG. 15A). At the end of the third round, the phages recovered presented a titter of 10⁵ (phages/mL), reflecting a high enrichment of the phages with increased ability to reach the BBB.

In contrast, no enrichment was observed when M13 helper phage was used as a control (FIG. 15A). Thus, the implemented in vivo phage display approach enabled the selection of sdAbs in a high stringency and restrictive environment that allowed a simultaneously subtractive selection of non-specific sdAbs.

### 3.3. Screening for Antibodies Towards the BBB

To characterize the diversity of the enriched sdAb population and identify the dominant clones, individual colonies recovered from the second and third in vivo selections were randomly picked and analysed by Sanger sequencing. Bioinformatic analysis of 64 sequenced clones (SEQ. ID. 44 to SEQ. ID 108) revealed the presence of 27 distinct groups, six of which included four or more representatives.

The most prevalent sequences would be repeatedly picked, yet the limitation in terms of simultaneous sequencing process hampers sanger sequencing from giving a wider overview of a 105 phage display library. To get further insights of the enriched sequences a next generation sequencing (NGS) of the third biopanning repertoire (FIG. 4B) was performed. NGS allows massive sequence analysis of the panning population, enabling a genomic assessment of the library diversity and frequency of each clone.

Following NGS analysis and subsequent sequencing filtering, we obtained 65, 701 sequences from the 2 min and 35, 472 sequences from the 60 min selection panning. This decrease of the V_{L} fragment diversity at the 60 min timepoint may be due to the increased stringency of the selection process. The percentage of singletons (sequences represented by only one count) was similar in both libraries, namely, 42.2% for the 2 min and 46.3% for the 60 min library of the total sequences. Sequence comparison of the main clones recovered from each biopanning demonstrated that the lead sequences were identical in terms of prevalence in both time points, although with a higher occurrence at the 60 min repertoire (N=5336, representing 15.0% of the total sequences) compared with the 2 min (N=4548, representing 6.9% of the total sequences) (FIG. 15B). Interestingly, among the top sequences from each time point, a higher sequence conservation was observed in the complementary determining regions (CDR), with most differences being allocated mainly at the antibody family level and frameworks (FR) regions.

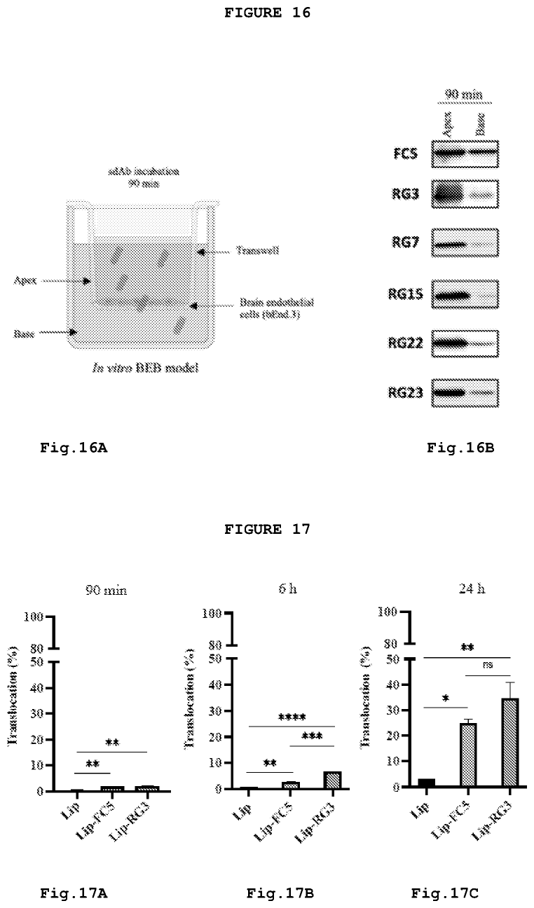

Following sequencing analysis, representatives of the most prevalent clones were harvested and characterized in terms of antibody expression and solubility). The most stable and predominant clones, RG3, RG7, RG15, RG22 and RG23, respectively SEQ. ID 44 to SEQ. ID. N. 48), were chosen further for expression, purification, and characterization in terms of BEB translocation. The ability of individual clones to transpose the BBB was first assessed in the in vitro BEB model. This model allowed us to understand the behavior, stability, and penetrability of each selected sdAbs in a cellular environment (FIG. 16A). Briefly, each clone was added to the apical chamber and incubated for 90 min. At the end of each assay, the apical and base volumes were collected and analysed by WB. In the present study a direct comparison of the selected BBB targeting sdAbs was performed with the VHH FC5, a promising camelid antibody selected from a nonimmunized phage library, that targets TMEM30A. All selected clones were able to translocate the bEnd.3 cell monolayer as demonstrated in FIG. 16B, with clones RG3, RG22 and RG23 showing an increased in vitro BEB-crossing efficiency. This assay allowed us to confirm the selection of highly competent brain targeting and BBB transposing sdAbs. A key feature of a potential antibody-based therapy is clearly its targeting ability. This is even more important for CNS approaches due to the intrinsic selectivity of the BBB. With the aim of demonstrating the BBB targeting and translocation ability of the selected antibodies and to determine the brain accumulation of the best clones, ^{99m}Tc(CO)₃-labelled individual sdAbs were intravenously injected in the tail vein of CD1 mice and the ex-vivo radioactivity of individual organs was measured. The biodistribution results of the tested clones at 2 and 60 min p.i. are presented in Table 1 and expressed as percentage of injected activity per gram of organ (% I.A./g±SD). The total radioactivity excretion is also presented as percentage of the total injected activity (% I.A.). For the sake of comparison, Table 1 includes also the biodistribution profile for the ^{99m}Tc(CO)₃-labeled FC5, a control antibody.

Table 1. Presents the biodistribution profiles of ^{99m}Tc(CO)₃-labeled sdAbs. To validate that the selected clones from phage display selection were the most competent sdAbs in terms of BBB translocation, an in vivo biodistribution assay was performed. SdAbs were radiolabelled with ^{99m}Tc(CO)₃(H₂O)₃ and intravenously injected in the tail vein of CD1 mice. Mice were sacrificed by cervical dislocation at 2 and 60 min p.i. and the radioactivity of each organ measured in a dose calibrator. The uptake in the brain and tissues of interest was calculated and expressed as a percentage of injected activity per gram of tissue (% I.A./g.)

TABLE 1
Biodistribution profiles of ^{99m}Tc(CO)₃-labeled sdAbs.

| | RG3-^{99m}Tc | | RG7-^{99m}Tc | | RG15-^{99m}Tc | | RG22-^{99m}Tc | | RG23-^{99m}Tc | | FC5-^{99m}Tc | |
|---|---|---|---|---|---|---|---|---|---|---|---|---|
| Organ | 2 min | 60 min | 2 min | 60 min | 2 min | 60 min | 2 min | 60 min | 2 min | 60 min | 2 min | 60 min |
| Blood | 10.1 ± | 1.4 ± | 11.2 ± | 1.5 ± | 12.5 ± | 3.0 ± | 13.2 ± | 0.8 ± | 7.8 ± | 1.1 ± | 11.0 ± | 1.0 ± |
| | 2.6 | 0.1 | 2.4 | 0.6 | 2.4 | 0.1 | 2.5 | 0.1 | 0.2 | 0.8 | 2.4 | 0.20 |
| Liver | 5.0 ± | 1.9 ± | 3.8 ± | 1.0 ± | 6.0 ± | 3.6 ± | 4.6 ± | 1.7 ± | 5.3 ± | 1.9 ± | 10.0 ± | 12.40 ± |
| | 0.9 | 0.3 | 0.8 | 0.4 | 1.0 | 0.7 | 0.7 | 0.2 | 0.2 | 0.4 | 1.5 | 0.4 |
| Intestine | 1.8 ± | 0.7 ± | 1.8 ± | 0.21 ± | 1.8 ± | 0.4 ± | 2.0 ± | 0.17 ± | 1.6 ± | 0.4 ± | 0.9 ± | 0.79 ± |
| | 0.6 | 0.1 | 0.2 | 0.08 | 0.4 | 0.1 | 0.2 | 0.03 | 0.7 | 0.1 | 0.4 | 0.04 |
| Spleen | 2.2 ± | 0.6 ± | 3.6 ± | 0.5 ± | 3.5 ± | 4.8 ± | 2.6 ± | 0.54 ± | 2.45 ± | 0.8 ± | 4.2 ± | 4.2 ± |
| | 0.6 | 0.1 | 0.6 | 0.3 | 1.2 | 1.7 | 0.6 | 0.03 | 0.04 | 0.1 | 0.8 | 0.80 |
| Heart | 3.2 ± | 0.4 ± | 4.7 ± | 0.45 ± | 4.3 ± | 1.3 ± | 3.2 ± | 0.29 ± | 4.5 ± | 0.5 ± | 3.9 ± | 0.76 ± |
| | 1.2 | 0.1 | 1.2 | 0.08 | 0.8 | 0.2 | 0.5 | 0.03 | 0.4 | 0.1 | 0.5 | 0.03 |
| Lung | 7.5 ± | 1.1 ± | 9.8 ± | 1.5 ± | 9.9 ± | 2.4 ± | 6.7 ± | 2.1 ± | 6.0 ± | 1.7 ± | 12.6 ± | 38.20 ± |
| | 2.7 | 0.2 | 2.4 | 0.7 | 1.2 | 0.4 | 0.6 | 0.4 | 0.9 | 0.3 | 0.6 | 6.20 |
| Kidney | 74.6 ± | 170.6 ± | 73.6 ± | 93.1 ± | 54.6 ± | 52.1 ± | 55.5 ± | 49.3 ± | 27.2 ± | 74.3 ± | 59.8 ± | 96.40 ± |
| | 9.9 | 19.1 | 7.5 | 6.9 | 7.5 | 9.0 | 0.9 | 8.3 | 4.1 | 10.7 | 2.4 | 6.80 |
| Muscle | 2.0 ± | 0.4 ± | 1.9 ± | 0.29 ± | 1.5 ± | 0.6 ± | 1.4 ± | 0.3 ± | 1.5 ± | 0.34 ± | 1.1 ± | 0.39 ± |
| | 1.0 | 0.2 | 0.2 | 0.07 | 0.4 | 0.1 | 0.1 | 0.1 | 0.2 | 0.05 | 0.1 | 0.04 |
| Bone | 2.7 ± | 0.81 ± | 2.3 ± | 0.33 ± | 2.3 ± | 1.0 ± | 2.4 ± | 0.5 ± | 1.5 ± | 0.4 ± | 1.7 ± | 0.80 ± |
| | 0.2 | 0.05 | 0.4 | 0.05 | 0.8 | 0.1 | 0.4 | 0.1 | 0.4 | 0.1 | 0.2 | 0.3 |
| Stomach | 1.0 ± | 0.97 ± | 1.7 ± | 0.9 ± | 0.7 ± | 1.2 ± | 0.7 ± | 0.26 ± | 1.1 ± | 0.9 ± | 1.2 ± | 0.80 ± |
| | 0.4 | 0.04 | 0.6 | 0.4 | 0.3 | 0.1 | 0.3 | 0.07 | 0.5 | 0.4 | 0.2 | 0.3 |
| Brain | 0.82 ± | 0.06 ± | 0.46 ± | 0.4 ± | 0.61 ± | 0.08 ± | 0.60 ± | 0.03 ± | 0.49 ± | 0.04 ± | 0.52 ± | 0.07 ± |
| | 0.05 | 0.02 | 0.1 | 0.1 | 0.1 | 0.06 | 0.15 | 0.01 | 0.02 | 0.02 | 0.18 | 0.02 |
| Excretion | — | 19.6 ± | — | 21.1 ± | — | 27.5 ± | — | 70.3 ± | — | 27.5 ± | — | 9.60 ± |
| (% I.A.) | | 5.4 | | 9.0 | | 5.1 | | 3.5 | | 5.1 | | 1.40 |

Total radioactivity excretion was expressed as percentage of injected activity (% I.A.). Statistical analysis was performed with one-way ANOVA, with p-value < 0.05 considered as the level of statistical significance.

Statistical analysis of the results indicated that at 2 min p.i., there was no significant differences between the clones under evaluation and the FC5 in most organs and tissues (blood, intestine, spleen, heart, muscle, bone and stomach), which reflects the short time point after administration. However, significant differences were found in organs related to the excretory paths (liver and kidneys) as well as in the lung and brain uptake. In fact, all tested clones presented a brain accumulation >0.4% I.A./g at 2 min p.i., with clones RG3 and RG15 displaying the highest brain accumulation and reaching values 0.82 and 0.61% I.A./g, respectively.

Both radiolabelled sdAbs predominantly accumulated in the kidneys, which may be related to their preferential renal excretion path and tubular reabsorption. At 60 min p.i., significant differences between the clones and FC5 were found in the radioactivity uptake in all evaluated organs and tissues except the brain. Significant differences were also found in the rate of total excretion.

As regards to brain uptake, the biodistribution data confirmed a lower accumulation at 60 min p.i., showing that sdAbs translocated the BBB and were rapidly recirculated back into the blood and excreted from the animal body and possibly indicating the presence of a receptor on the luminal and basal side of the endothelial cells. This non-retention effect strengthens the potential of the selected sdAbs as drug delivery vectors that may circumvent the brain accumulation issue and its derived toxic effects. Among the selected clones for in vivo testing, four out of the five ^{99m}Tc(CO)₃-labelled sdAbs presented similar or superior brain accumulation compared to FC5. Within the panel of selected sdAbs, a superior brain accumulation was observed for the RG3 clone, (0.82±0.05% I.A./g) and, as far as we are aware, positioning as one of the most competent sdAb in BBB translocation described so far.

### 3.5. Validation of the RG3 as a BBB Drug Delivery Vector

A major goal of our study was also to explore the potential of our selection platform to distinguish nano-antibodies for targeted drug delivery approaches. To achieve this, the most competent sdAb, RG3, along with the control FC5, were engineered at liposome surface encapsulating the HDACi PAN, and its BBB-transmigrating properties and antitumour al activity were validated in a dual functional in vitro BEB-glioblastoma model. Glioblastoma is an aggressive brain tumour highly resistant to chemotherapy, with very limited therapeutic strategies due to the poor drug penetration through the BBB. Among the promising strategies for cancer treatment, PAN has emerged as a highly efficient new class of anticancer drug. Nevertheless, PAN, as many other chemotherapeutics, does not cross the BBB.

Among the wide panel of nanoparticles, liposomes are on the front line of nanocarrier based strategies for glioma therapy. Liposomes are lipid vesicles constituted by one or more concentric lipid bilayers separated by compartments, to aqueous Due their unique characteristics, incorporation of both hydrophobic and hydrophilic compounds, along with its biocompatibility, prolonged circulation time, sustained drug delivery and ability to be conjugated with a targeting moiety, liposomes have a remarkable potential as brain-targeted carrier systems. In addition, the nine FDA approved liposomal based formulations for cancer therapy further supports the therapeutic potential of this lipid base carriers. Therefore, liposomes encapsulated with PAN and conjugated with our BBB-sdAbs can be used as a novel targeted drug delivery system.

Accordingly, liposomes loaded with PAN were successfully developed, with a mean size of 110 nm and an encapsulation efficiency of 65±2% using the lipid composition DPPC:Chol:DSPE-PEG:DSPE-PEG-Biotin at a molar ratio of 1.85:1:0.14:0.01. No significative differences in terms of encapsulation efficiencies and vesicle sizes were observed among biotinylated and non-biotinylated liposomes.

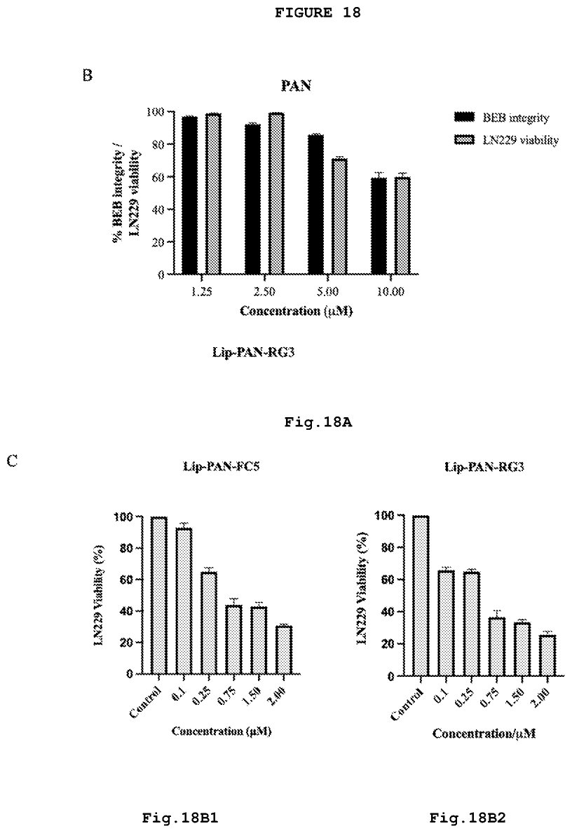

To assess the cellular cytotoxicity of the unconjugated and BBB-sdAbs conjugated PAN-loaded liposomes against glioblastoma, a cell viability assay was carried out with the glioblastoma cell line LN229. Similar to the free PAN formulation, PAN loaded liposomes exhibited a potent activity and dose-dependent inhibitory effect on the proliferation of LN229 cells). Moreover, the antibody conjugation at liposomes surface did not affect the inhibitory effects of PAN loaded liposomes on the proliferation of LN229 cells and no significant differences between liposomes conjugated with FC5 or RG3 were observed. In contrast, no cytotoxicity activity was observed for PAN free RG3 and FC5 liposome formulations (Lip-RG3 and Lip-FC5). In parallel, the translocation efficiency of the Lip-RG3 and Lip-FC5 in the in vitro monocellular model was evaluated. Here, RG3 and FC5 functionalized rhodamine loaded liposomes were added to the apical side of the model and collected following a 90 min, 6 and 24 h incubation, reaching a translocation maximum at the model base of 29.3±6.5% at 24 h in the case of RG3-liposome, 9.4 times higher than the non-conjugated liposomal formulation. In turn, the percentage of translocation for the FC5 conjugated liposome was 12.9±1.6%, only 4.2 times higher than the non-conjugated liposomal formulation (FIG. 17). In addition, monitorization of BEB integrity demonstrated that both liposomal formulations had no significant impact at the barrier structure. Finally, the antiproliferative activity of the PAN loaded in RG3 and FC5 conjugated liposomes following BBB translocation was determined in a dual functional in vitro BEB-glioblastoma model (BEB-GBM) (FIG. 18). To develop the BEB-GBM model, a non-contact co-culture system composed of bEnd.3 as the BBB barrier and the LN229 cell line at the base as the GBM cell line was implemented. To evaluate the cytotoxicity on bEnd.3 cells and determine the most appropriate PAN concentration to be tested in the BEB-GBM model, increasing concentrations of free PAN were added to the apex of the BEB-GBM model and its effect in the BEB integrity and LN229 cytotoxicity was determined. As shown in FIG. 18A, no significant effect in the BEB integrity and LN229 viability was observed when PAN free was added below 2.5 M. Thus, the BEB translocation efficiency and drug delivery of the PAN encapsulated in RG3 and FC5 functionalized liposomes was assessed by adding liposomes to the BEB-GBM model apex following 24 h incubation at different concentrations (0.1-2.0 μM). As shown in FIG. 18B1 and B2, both RG3 and FC5 functionalized PAN liposomes were able to translocate the in vitro BEB-GBM model and exhibited a dose-dependent inhibitory effect in the LN229 cell line without influencing the overall barrier integrity of the monocellular model.

To corroborate that the cytotoxic effects of the RG3 and FC5 conjugated liposomes on GBM cell line were related to histone acetylation induction, the key molecular mechanism of HDACis, the H3 acetylation status of cells treated with PAN-loaded in RG3 and FC5 conjugated liposomes was compared with the H3 acetylation status of unloaded liposome formulations and vehicle/control treated cells. Immunoblotting analysis demonstrated that LN229 GBM cell line presented an hyperacetylation status following 24 h treatment with both RG3 and FC5 PAN-loaded liposomes, when compared with unloaded liposomes and vehicle/control treated cells. A final in vivo proof-of-concept biodistribution study was performed to demonstrate the BBB translocation of our RG3 functionalized liposome system. For that, ¹¹¹indium radiolabelled RG3 functionalized liposomes were prepared and administered to CD1 mice.

As shown in Table 2, 2 min after intravenous injection (i.v.), 1% of the injected dose per gram of tissue (% ID/g), of our developed radiolabelled system, has reached the brain and this amount was constant 60 min after administration. In contrast, a significantly lower brain uptake was observed for the unconjugated liposome. Statistical analysis of the biodistribution and excretion data at 1 and 24 h p.i., between the RG3-conjugated liposomes and the control liposomes indicate significant differences in almost all studied organs and tissues at 1 h p.i., except the intestines and stomach. Indeed, a significantly higher activity was found in the blood stream and a higher uptake in most organs. A significantly lower rate of total radioactivity excretion of the RG3 conjugated liposomes (15.4±2.5% and 28.2±2.7% I.A. at 1 h and 24 h, respectively versus 59.7±6.6% and 68.3±0.4% for the control liposomes). At 24 h p.i. the significant differences were maintained for the main organs namely blood, brain, excretory organs (kidneys, liver, intestines) and total radioactivity excretion.

Altogether, the in vivo and in vitro data have shown that our selected BBB-sdAbs and the developed sdAb-liposome system have a high ability to cross the BBB and are an advantageous strategy for CNS-targeted therapies. Table 2 presents the biodistribution of selected RG3 conjugated liposome. To validate the BBB translocation of the RG3-conjugate liposome an in vivo biodistribution assay was performed. RG3-conjugated liposomes were radiolabelled with ¹¹¹In and intravenously injected of the tail vein of CD1 mice. Mice were sacrificed by cervical dislocation at 2 min, 60 min and 24 h following injection and the radioactivity of each organ measured using a dose calibrator. The uptake in the brain and tissues of interest was calculated and expressed as a percentage of injected radioactivity dose per gram of tissue (% ID/g). Total radioactivity excretion was expressed as percentage of injected activity (% I.A.).

TABLE 2
Biodistribution of selected RG3 conjugated liposome

| | ¹¹¹In-Lip-RG3 | | | ¹¹¹In-Lip-(Control) | |
|---|---|---|---|---|---|
| Organ | 2 min | 60 min | 24 h | 60 min | 24 h |
| Blood | 33.0 ± 7.4 | 26.2 ± 8.1 | 8.9 ± 1.7 | 12.7 ± 3.8 | 3.5 ± 0.6 |
| Liver | 8.1 ± 2.8 | 10.7 ± 2.1 | 11.7 ± 1.6 | 1.1 ± 0.6 | 5.9 ± 0.5 |
| Intestine | 0.4 ± 0.1 | 0.9 ± 0.4 | 2.3 ± 0.1 | 0.55 ± 0.06 | 0.98 ± 0.07 |
| Spleen | 4.3 ± 0.8 | 19.4 ± 3.2 | 18.2 ± 0.1 | 1.7 ± 0.4 | 12.5 ± 4.2 |
| Heart | 4.2 ± 0.8 | 4.4 ± 1.2 | 3.0 ± 1.3 | 0.7 ± 0.2 | 1.3 ± 0.3 |
| Lung | 13.3 ± 3.9 | 10.2 ± 1.0 | 3.9 ± 1.6 | 1.0 ± 0.5 | 1.6 ± 0.6 |
| Kidney | 7.0 ± 1.7 | 6.6 ± 1.0 | 6.4 ± 0.7 | 1.3 ± 0.6 | 2.7 ± 0.3 |
| Muscle | 0.8 ± 0.2 | 0.62 ± 0.07 | 0.9 ± 0.2 | 0.4 ± 0.1 | 1.3 ± 0.9 |
| Bone | 1.8 ± 0.1 | 1.6 ± 0.2 | 1.1 ± 0.9 | 0.6 ± 0.3 | 0.7 ± 0.3 |
| Stomach | 0.8 ± 0.2 | 0.8 ± 0.6 | 0.8 ± 0.2 | 1.7 ± 0.9 | 1.1 ± 0.2 |
| Brain | 1.0 ± 0.3 | 1.0 ± 0.4 | 0.4 ± 0.2 | 0.13 ± 0.03 | 0.16 ± 0.07 |
| Carcass | 45.2 ± 1.8 | 43.6 ± 4.8 | 38.3 ± 3.6 | 18.0 ± 0.9 | 26.0 ± 2.3 |
| (% I.A.) | | | | | |

Statistical analysis of the data was performed (t-test), with p-value<0.05 considered as the level of statistical significance.

### In Conclusion

The present invention shows that with an immunized rabbit derived sdAb library developed towards BBB epitopes and an in vivo phage display selection method, a panel of nano-antibodies with one of the highest levels of BBB translocation described so far was identified. Moreover, the developed RG3-liposome specifically targets and translocates the BBB, delivering a payload, in in vitro settings, at effective concentrations and constituting a strong candidate for drug delivery to the CNS. Hence, the proposed in vivo sdAb development platform is a pioneering selection process of highly specific nano-antibodies with promising properties for brain targeting and drug delivery to different CNS diseases, such as brain tumours, Alzheimer's, or Parkinson diseases.

### EXAMPLES

### Example 1. Canine Multicentric Lymphoma Biobank

Patients with canine multicentric lymphoma were followed at the oncology unit of the Veterinary Medicine Faculty—University of Lisbon (FMV/UL)'s—Teaching Hospital, where clinical evaluations were conducted. On a preliminary phase, with diagnostic and staging purposes, a complete history, clinical signs and physical examination were assessed. Complete blood count and biochemistry profile were performed, as well as abdominal thoracic imaging exams. Histopathological evaluation of lymph nodes was performed after node biopsy. This histopathological evaluation included a morphologic examination, classification of lymphoma into grade subcategories and immunophenotyping to determine the immunophenotype present—B or T immunohistochemistry markers included CD3, CD20, CD79αcy and PAX-5. This clinical and laboratory examination allowed staging the dogs using the World Health Organization (WHO) system.

Inclusion criteria comprised dogs recently diagnosed with multicentric lymphoma by clinical examination and cytological examination of lymph node fine-needle aspirate that have not yet begun therapy. Exclusion criteria included dogs who have begun chemotherapy and who have received steroids or other immunotherapeutic agents within the last eight weeks of study enrolment or dogs who have become severely ill.

All sample collection was conducted with written pet owner consent in accordance with the principles and procedures outlined in the NIH Guide for the Care and Use of Animals and approved by the Animal Care and Use Committee of FMV/UL.

Blood samples allowed the isolation of plasma and serum, as well as the extraction of DNA (Dneasy Blood & Tissue, Qiagen, Hilden, Germany) and Mrna (Rneasy Protect Animal Blood System, Qiagen), that were stored at −80° C.

Additionally, PBMC were isolated by Ficoll gradient method (Biocoll Separating Solution, BioChrom®, Fisher Scientific, New Hampshire, USA) and following cell viability assessment, aliquots of 5×10⁶ cells were suspended in 90% Foetal Bovine Serum (FBS) (Gibco, Life Technologies, Paisley, UK) and 10% dimethyl sulfoxide (DMSO) (Sigma-Aldrich, Missouri, USA) and kept in liquid nitrogen.

Sterile biopsy lymph nodes samples were divided, ⅓ was fined cut and stored at −80° C. in RNAlater® (Invitrogen, Life Technologies, Paisley, UK), ⅓ was formalin-fixed and ⅓ stored in liquid nitrogen after lymphoma cell isolation. Briefly, solid tissue was cut, passed through a cell strainer (Cell Strainer, BD Falcon®), suspended in Roswell Park Memorial Institute-1640 (RPMI-1640) medium (Gibco) supplemented with 20% FBS and penicillin 100 U/ml plus streptomycin 0.1 mg/ml (Gibco), and isolated through Ficoll gradient method (Biocoll Separating Solution, BioChrom®).

Cell viability was assessed and for storage purposes, aliquots of 5×10⁶ cells were suspended in 90% FBS and 10% DMSO and kept in liquid nitrogen. Clinical follow-up information about all cases was gathered from electronic medical records. All dogs that participated in this study were client-owned animals which joined the study during their diagnostic assessment. All sampled animals stayed with their owners after sample collection.

### Example 2. Cell Lines and Culture

The canine B-cell lymphoma cell line CLBL-1 was provided by Dr. Barbara Rütgen (University of Vienna, Austria). The human Burkitt's lymphoma Raji cell line, the human T lymphocyte cells and the human cell line HEK293T cell line (appropriated for ectopic expression of mammalian proteins) were obtained from the American Type Culture Collection (ATCC, Manassas, VA). CLBL-1, Raji and Jurkat cell lines were maintained in RPMI-1640 medium (Gibco) supplemented with 10% FCS (Gibco) and penicillin 100 U/ml/streptomycin 0.1 mg/ml (Gibco). HEK293T cell line was cultured in DMEM medium supplemented with 10% FCS (Gibco) and penicillin 100 U/ml/streptomycin 0.1 mg/ml (Gibco). All cell lines cultures were maintained at 37° C. in a humidified atmosphere of 5% CO₂ (T75-tissue culture flasks, Greiner Bio-One, Kremsmünster, Austria).

### Example 3. Rabbit Immunization with cNHL Primary Cells

Three female New Zealand White rabbits (Charles River) were immunized and boosted for 4 months with 1×10⁷ of cNHL primary cells from our biobank to induce a strong and specific immune response against NHL receptors. Patient 5 and patient 6 cells from our biobank diagnosed with Diffuse Large B Cell Lymphoma (DLBCL) were selected. For that purpose, tumour cells isolated from lymphoma affected lymph nodes were thawed, washed in PBS and after confirmation of cell viability, resuspended in 1 ml of PBS. The injections were administered subcutaneously at 2 weeks intervals. Before each immunization blood was harvested from the marginal ear vein for serum isolation. Five days after the final boost, rabbits were sacrificed by cardiac puncture exsanguination, following propofol anaesthesia, and spleen and bone marrow were harvested for total RNA isolation and Cdna synthesis.

### Example 4. Characterization of Rabbit Immune Response

The rabbit immune response developed against the biobank cNHL primary cells and CLBL-1 cells was monitored by ELISA sera testing. Pre-bleed sera was used as control. Briefly, 50×10³ cells were blocked with PBS-BSA 1% (BSA, bovine serum albumin, Merck) for 30 min, washed with PBS and incubated with serial dilutions of the rabbit serum (from 1/1000 to 1/32000) for 1 h. Cells were then washed with PBS and secondary antibody goat-a anti-rabbit IgG-Fc specific HRP (Jackson ImmunoResearch) at 1:3000 in PBS-BSA 1% was added to each well and incubated for 1 h. Following incubation, ABTS substrate solution (Merck) was added, and optical density (OD) was measured with a microplate reader (Bio-Rad) at 405 nm.

Each serum was also analysed for its binding properties against Cnhl cells by FACS. For that, CLBL-1 cells and Cnhl cells from patients 5 and 6 were prepared. Cells were washed twice in PBS-BSA 0.5% and incubated with the rabbits' pre-bleed and final bleed (1:3000) 30 min at 4° C. Cells were then washed with cold PBS-BSA 0.5% 3 times and incubated with secondary antibody (Alexa Fluor® 647 Goat Anti-Rabbit IgG) at 1:10000 in PBS-BSA 0.5% for 30 min at 4° C. Cells were washed with cold PBS-BSA 0.5% 3 times and submitted to FACS analysis (FACSCalibur).

Unstained cells were used as negative control for voltage settings. For multiple-colour sorts, single colour controls were used for compensation settings. Data was analysed by FlowJo software version 10 (FlowJo LLC).

To evaluate the protein profile recognized by the rabbit serum, immunoblotting was performed using CLBL-1 cells, Cnhl primary cells (B1 and B2) and PBMC from healthy dogs (C1 and C2). Peroxidase-conjugated goat anti-rabbit antibody (Jackson Immune Research) was used as secondary antibody.

### Example 5. Construction of Single Domain Antibody Library

Total RNA was extracted from the spleen and bone marrow of each rabbit using Trizol reagent according to the manufacture instructions (Invitrogen). First-strand Cdna was synthesized using Transcriptor High Fidelity (Roche) following the manufacturer's instructions. The first strand cDNAs from each rabbit were then subjected to separate 30-cycle polymerase chain reactions using Phusion High Fidelity DNA polymerase (Thermo Fisher Scientific) and 10 specific oligonucleotide primer combinations for the amplification of rabbit sdAbs in the format of the light chain variable region (V_{L}) (9×V_{κ} and 1×V_{λ}) as previously described. PCR products encoding a library of antibody fragments (sdAbs) were then gel purified, restriction digested with SfiI and cloned into Pcomb3Xss. Subsequently, the ligated product was transformed into electro competent cells via electroporation and the library was tittered. To confirm library insert efficiency and diversity, PCR colony was performed using primer RSC-F and primer RSC-B. Phage library sequencing was performed by GATC Biotech AG (Ebersberg, Germany) using the pComb3x ATG primer. To translate to amino acid sequences and to evaluate homology, the Vector NTI Advance 10 software (Thermo Fisher Scientific) was used.

### Example 6. Phage Display Selection of Antibodies Targeting cNHL

The phage library displaying V_{L} sdAbs was first panned using a subtractive cell phage display protocol as previously described by Carlos Barbas and our studies (Barbas III, C. F., Burton, D. R., Scott, J. K. & Silverman, G. J. Phage Display: A Laboratory Manual. (Cold Spring Harbor Laboratory Press, 2001) and Dias, J. N. R. et al. Characterization of the canine CD20 as a therapeutic target for comparative passive immunotherapy. Sci Rep 12, 2678 (2022).), and that included a negative selection on HEK 293T cells followed by a positive selection on CLBL-1 cells. Then, after three rounds of in vitro selections, an additional panning was performed in vivo in a xenograft CLBL-1 murine model. Briefly, female 6-8-wk-old SOPF/SHO SCID mice (Charles River) were maintained in microisolation cages under pathogen-free conditions. Mice were allowed to acclimatize for at least two weeks prior to the start of the experiment. Then, 1×10⁶ CLBL-1 cells in PBS with matrigel (Corning, NY, USA) (1:1) were injected subcutaneously into the dorsal interscapular region to induce tumours. When tumours reached a minimum volume of 100 mm³, three SCID mice were intravenously injected into the tail vein with 100 μl of phage (1×10¹⁰ pfu/ml) freshly prepared from the third in vitro selection round. Phages were allowed to circulate for 60 min, then mice were sacrificed, perfused and xenograft tumours were removed and weighted. Following tumour homogenization in 70 μm cell strainers (VWR, Radnor, PA, USA) phages were recovered by incubating the homogenized tumour with 500 μl of freshly prepared trypsin (1 mg/ml) (Gibco), supplemented with anti-protease (Merck) and DNAse (1 U/μL) (Invitrogen) for 15 min at 37° C. Then, the eluted phages (binders) were recovered after a centrifugation at 10,000×g for 10 min at 4° C. and normalized to a final volume of 1 mL in PBS. To elute the internalizing phages, the cell pellet obtained after the trypsin elution was washed 3× with PBS and centrifuged at 10,000×g at 4° C., 5 min. Then, the cell pellet was resuspended with 200 μl of 0.1 M triethylamine, incubated 10 min and neutralized with 50 μl of 1 M Tris 7.5. The eluted phages were normalized for a final volume of 1 ml. Each output phage (binders and internalizers) obtained was used for phage titration and re-amplification in Escherichia coli ER2738 (Lucigen) cells for storage and lead selection. Phages were also tittered in blood. An irrelevant naïve rabbit V_{L} sdAb library and the M13 helper phage were used as controls in a pilot study.

### Example 7. ELISA Screening of Antibodies Targeting cNHL

To express and select anti-NHL-sdAbs, phagemid DNA encoding selected anti-cNHL sdAbs was cloned into PT7-PL (PT7-peptide leader) vector and transformed into E. coli strain BL21. Individual colonies were inoculated in 100 μl of Super Broth (SB) medium containing Overnight Express™ Autoinduction System (Novagen®) and 100 μg/ml of ampicillin and incubated overnight at 30° C. Next day, 40 μl of BugBuster (Roche) containing anti-protease cocktail-EDTA free inhibitors (Roche) were added and incubated for 30 min at 4° C. Then, plates were centrifuged at 1200 rpm and the supernatant was tested in ELISA assays. Three different conditions were assessed: binding to the sdAbs to the antigen, expression level and unspecific binding. After coating the wells with CLBL-1 cell extracts or Raji cell extracts for 1 h at 37° C., wells were blocked with 3% BSA in PBS. Then, wells were washed with PBS and clones were added and incubated for 1 h, at 37° C. Next, plates were washed and incubated with anti-HA HRP antibody (Roche). Finally, after 1 h incubation, plates were washed and ABTS (Roche) was added and optical density at 405 nm was measured at different time points. To evaluate the expression level, the same protocol was applied, excluding antigen coating.

For unspecific binding, antigen was replaced by 3% BSA. Rabbit sera and anti-CD20 antibody were used as positive controls. BL21 cell extracts were used as negative controls. In the end, the ten best individual clones were sequenced at Eurofins company. Sequence analysis was performed using the Vector Nti software (Invitrogen). The antibody frameworks, CDRs and the amino acid numbering and sequences followed the rules described by Kabat et al. (1991) Sequences of proteins of immunological interest. Bethesda, MD, U.S. Dept. of Health and Human Services, Public Health Service, National Institutes of Health. Sequences obtained were compared and aligned with the NGS data using the Vector NTI software (Invitrogen), as described above.

### Example 8. Analysis of Phage Display Enrichment and Next Generation Sequencing

To analyse the amino-acid sequence and profile of the selected clones by ELISA, the 43 best individual clones were sequenced at Eurofins company. Sequence analysis was performed using the Vector NTI software (Invitrogen). The antibody frameworks, CDRs and the amino acid numbering and sequences followed the rules described by Kabat et al., as above indicated.

Additionally, to assess diversity and enrichment achieved by phage display, we performed next generation sequencing (NGS). For such, we used the 250-paired ended module of the Miseq (Ilumina) sequencing platform to obtain the whole sequence of the VL sdAb regions of the initial library and the in vivo biopanning. The Miseq library for sequencing was set up by amplifying the VL sdAb regions. Then, 2 μg of the purified amplicons were sent for sequencing at STABVIDA company. The data was analysed using the Geneious software.

Data obtained was assembled by merging the paired-ended sequence reads, translating the sequence into protein and discharging all sequences with less than 100 amino acids, no Sfi cut site and histidine tail sections. Later, a custom python script was developed to organize and count the sequence reads. In the end, sequences obtained were compared and aligned with the NGS data using the Vector Nti software (Invitrogen), as described above. The data was represented by a graph, where the pattern of sequence reads was shown.

### Example 9. Production and Purification of sdAbs

The three best sdAbs were selected according to NHL cell binding capacity and one (C5I) of those was expressed and purified. To express and purify the C5I clone, DNA cloned in a PET21 expression vector (Sigma-Aldrich, St. Louis, MO, USA) was transformed into nonsuppressor E. coli strain BL21 (DE3) (Lucigen, Midddleton, WI, USA). C5I was produced by inoculating 10 μl of the frozen clone in Super Broth (SB) medium containing 100 g/ml of ampicillin. The culture was grown overnight at 37° C. and then diluted 1:30 in SB medium, 100 μg/ml ampicillin. When the culture reached OD_{600 nm}=0.6, clone expression was induced by the addition of 0.6 Mm isopropyl-1-thio-β-D-galactoside (IPTG) and incubated overnight at 19° C. After expression, bacteria were harvested by centrifugation (4000 rpm, 15 min, 4ºC), and resuspended in 50 ml of initial buffer (50 Mm HEPES, 1 M NaCl, 10 Mm Imidazole, 2 M Urea, 5 Mm CaCl₂, 1 Mm β-mercaptoethanol, and Ph=8) supplemented with protease inhibitors (Roche). Cells were lysed by sonication and the inclusion bodies were recovered by centrifugation (9000 rpm, 30 min, 4° C.).

The pellet was washed (50 mM HEPES, 1 M NaCl, 10 mM Imidazole, 2 M Urea, 5 Mm CaCl₂, 1 Mm β-mercaptoethanol, and Ph=8), sonicated and centrifuged (9000 rpm, 30 min, 4ºC). Then, the inclusion bodies were resuspended in a 6 M Urea buffer (50 Mm HEPES, 1 M NaCl, 10 Mm Imidazole, 6 M Urea, 5 Mm CaCl₂, 1 Mm β-mercaptoethanol, and Ph=8) and incubated overnight at 4° C., under agitation for protein denaturation. A final centrifugation step was performed to remove cellular debris, and the supernatant was filtered through a 0.2 μm syringe filter. The denatured sdAbs were purified by nickel chelate affinity chromatography using the C-terminal His tag. Bound proteins were eluted in high concentrated imidazole buffer (50 Mm HEPES, 1 M NaCl, 500 Mm Imidazole, 6 M Urea, 5 Mm CaCl₂, 1 Mm β-mercaptoethanol, and Ph=7.8). Refolding was performed by step wise dialysis, according to Gouveia et al, 201730. After that, C5I was purified by size exclusion chromatography (SEC) using HiPrep 16/60 Sephacryl S-100 column (Sigma-Aldrich). Protein purity was analysed by sodium dodecyl sulphate/polyacrylamide gel electrophoresis (SDS/PAGE) gel with 15% acrylamide gel under denaturing conditions.

### Example 10. Immunofluorescence Microscopy

1.5×10⁵ of CLBL-1 cells were plated on ibidi μ-Slide 8 Well Glass Bottom (#80827, Ibidi, Germany) and incubated for 24 h at 37° C. in a humidified atmosphere of 5% CO₂. Then, 5 μM of V_{L} was added to the cells and incubated for 90 minutes at 37° C. After incubation, cells were washed twice with PBS, fixed with PFA 4% for 15 min at RT, permeabilized with 0.1% Triton X-100 for 10 min at RT, washed, blocked with 0.1% triton X-100 and 3% BSA in PBS (blocking solution) and incubated overnight with anti-HA (Roche, 1:50) at 4° C. Next day, cells were washed twice with PBS and incubated with anti-rat Alexa Fluor-488 (1:500) for 1 h at RT. After washing, DAPI Vectashield (Vector Labs, CA, USA) was added to the cells. Image acquisition was performed on a confocal point-scanning Zeiss LSM 880 microscope (Carl Zeiss, Germany) equipped with a Plan-Apochromat DIC X63 oil objective (1.40 numerical aperture). Diode 405-30 laser was used to excite DAPI, and argon laser in the 488-nm line to excite Alexa Fluor-488. In the Airyscan acquisition mode, ×1.80 zoom images were recorded at 1024×1024 resolution. ZEN software was used for image acquisition and Fiji software was used for image processing.

### Example 11. Binding and Internalization Characterization of C5 V_{L} sdAb

The C5 V_{L} sdAb binding and cellular internalization properties towards CLBL-1 cells were studied by cytometry analysis and immunofluorescence. For cytometry analysis, C5 was incubated for 90 min with CLBL-1 cells as described in the material and methods section. The data shown in FIG. 5A demonstrated that C5 specifically binds to the CLBL-1 cells. On the contrary, no binding interaction of C5 with Jurkat cells was observed (FIG. 5B). Live/dead reagent was used to exclude dead cells and the background noise was also evaluated in the control with the secondary antibody (data not shown). To better characterize the binding of the C5 to the CLBL-1 cells, we further evaluated the internalization of the C5 sdAb on the cells using an immunofluorescence assay. As shown in FIG. 5C, a high density of Alexa Fluor-488 labelled C5 sdAb was observed in the perinuclear region. Conversely, there was no detectable fluorescence in the control sample image nor in Jurkat cells (FIG. 5D). Thus, these data confirmed the C5 binding to the CLBL-1 cells and its internalization into the cytoplasm. This cellular internalization feature is essential to develop an efficacious ADC.

### Example 12. Bioconjugation VL-DAB-SN38

1 μL of H₂O, 5 ML of compound DAB (4 Mm in DMSO) and 1 μL of IS (20 Mm in DMSO) were added to 993 μL of PBS Ph 7.4 with 10% DMSO to obtain a final solution of compound DAB-SN38 (20 Um, PBS Ph 7.4, 10% DMSO). To a PBS Ph 7.4 solution containing VL (10 μM) and TCEP (1.5 equiv., 3.5 Mm), DAB (20 equiv., 9 Mm, DMSO) was added and the solution was mixed during 1.5 h at 25° C. The expected conjugate was evaluated after 1.5 h by High-Resolution Mass Spectrometry, recorded in a Thermo Scientific Q Exactive hybrid quadrupole-Orbitrap mass spectrometer (Thermo Scientific™ Q Exactive™ Plus). The final immunoconjugate VL-DAB-SN38 was detected. The mass spectra were deconvoluted using MagTran software.

### Example 13. Cytotoxic Assay

To determine the effect of VL-DAB-SN-38 in CLBL-1 and Jurkat cell proliferation, a cell viability assay was performed using the Cell Proliferation Reagent WST-1 (Roche, Basel, Switzerland). Briefly, cells were seeded at a density of 6×10⁴ well in 200 μl of culture medium and subjected to increasing concentration (2.5 Mm to 12.5 Nm) of each compound (VL, VL-DAB-SN38 and SN-38). After 48 h treatment, cell viability was assessed using WST-1, following the manufacturer's instructions. Absorbance at 450 nm was measured using the iMark microplate Reader (Bio-Rad). Two replicate wells were utilized to determine each data point and three independent experiments were carried out in different days. Best-fit EC50 values of each formulation were calculated using GraphPad Prism software (version 8.0, San Diego, CA, USA) using the log (inhibitor)) vs response (variable slope) function.

### Example 14. DNA Topo I Activity Assay

Topo I activity on VL-DAB-SN38 was determined using the Human Topoisomerase I Assay Kit (Topogen, CO, USA) according to the manufacturer's instructions. Briefly, 30 μl of reaction containing the VL-DAB-SN38 were incubated with 1× reaction buffer (10 Mm Tris-HCL Ph=7.9, 1 Mm EDTA, 0.15 M NaCl, 0.1% BSA, 0.1 Mm Spermidine, 5% glycerol) and 10 U of Topo I for 1 h at 37° C. Then, the previously reaction mixture was incubated with supercoiled DNA for 1 h at 37° C. To stop the reaction, stop loading buffer (0, 125% bromophenol blue, 25% glycerol, 5% Sarkosyl) was added to the reaction. Samples were loaded on a 1% agarose gel and run in 1×TAE buffer. Then, gel was stained with ethidium bromide for 45 min and destained in distilled water. Relaxed DNA, SN38 and VL were used as controls.

### Example 15. Biodistribution Studies and Tumour Targeting

To evaluate the biodistribution and tumour targeting on a xenograft model of NHL, the selected V_{L} sdAb (C5) was radiolabeled with the radioactive precursor [^{99m}Tc(CO)₃(H₂O)₃]⁺ prepared from an IsoLink® kit (Covidien, Ireland). Radiochemical purity (RP) was checked by Reversed-phase high-performance liquid chromatography (RP-HPLC) and instant thin-layer chromatography silica gel (ITLC-SG, Agilent Technologies, USA). In brief, fac-[^{99m}Tc(CO)₃(H₂O)₃]⁺ solution was added to a nitrogen-purged closed glass vial containing a solution of His-tag with C5 to obtain a final concentration of 1 mg/ml. The mixture was incubated for 45-60 min at 37° C. and then a ITLC-SG analysis using 5% HCL (6M) solution in MeOH as eluent was performed to evaluate the RP of ^{99m}Tc(CO)₃—C5. While [^{99m}Tc(CO)₃(H₂O)₃]+ and [^{99m}TcO₄]⁻ migrate in the front of the solvent (Rf=1), the ^{99m}Tc(CO)₃—C₅ remains at origin (Rf=0). Radioactivity distribution on the ITLC-SG strip was evaluated using a miniGita Star scanning device (Elysia-Raytest, (Germany) coupled with a Gamma BGO-V-Detector (Elysia Raytest). For purification and concentration of the ^{99m}Tc-labeled sdAb, a 3 K Amicon (Merck Milipore) was used. ^{99m}Tc(CO)₃—C₅ diluted in PBS was used for the biodistribution studies after RP determination by ITLC-SG. For that, mice were intravenously injected in the tail vein with 100 μl of ^{99m}Tc(CO)₃—C₅ and sacrificed by cervical dislocation at 15 min, and 3 h after injection. Radioactivity was measured using a dose calibrator (Carpintec CRC-15W). After the removal of the tumour and tissues of interest, their radioactivity was measured using a γ-counter (Berthold, Germany). The uptake was represented as a percentage of injected activity dose per gram of organ or tissue (% ID/g).

### Example 16. Rabbit Immunization with BBB Cells

All animal-handling procedures were performed according to EU recommendations for good practices and animal welfare and were approved by the Animal Care and Ethical Committee. The animals were housed in a temperature and humidity-controlled room with a 12 h light-12 h dark cycle. Two New Zealand white rabbits (Charles River) were immunized and boosted at days 14, 28, 56 and 70, with mouse brain endothelial cells bEnd.3 cells (ATCC®CRL-2299™) to induce a strong and specific immune response against endogenous bEnd.3 receptors. Briefly, bEnd.3 cells were grown until confluency on T175 flasks in Dulbecco's Modified Eagle Medium (DMEM) media with high glucose and pyruvate (Gibco) supplemented with 10% heat inactivated fetal bovine serum (FBS) (Gibco) in a humidified atmosphere at 37° C. with 5% CO₂. Before each immunization procedure, blood was collected from the ear vein and 1×10⁶ bEnd.3 cells, suspended in 0.5-1 Ml of sterile phosphate buffer saline (PBS), injected subcutaneously in the rabbit. The injections were administered at 2-3-week intervals. Five days after the final boost, the rabbits were sacrificed by cardiac puncture exsanguination, following propofol anaesthesia, and spleen and bone marrow were harvested for total RNA isolation and cDNA synthesis.

### Example 17. Characterization of Rabbit Immune Response

The rabbit immune response developed against the bEnd.3 cells was monitored by ELISA sera testing of the bleeds taken before and after each boost injection. Briefly, 2×10⁴/well bEnd.3 cells were plated in a 96 well plate and incubated for 24 h at 37° C. in a humidified environment with 5% CO₂. In the following day, cells were blocked with PBS-BSA 1% (BSA, bovine serum albumin, Merck, Kenilworth, NJ, USA) for 30 min, washed with PBS and incubated with serial dilutions of the rabbit serum (from 1/500 to 1/32,000) for 1 h. Cells were then washed with PBS and secondary antibody goat-α anti-rabbit IgG-Fc specific HRP (Jackson ImmunoResearch, West Grove, PA, USA) at 1:3000 in PBS-BSA 1% was added to each well and incubated for 1 h. Following incubation, ABTS substrate solution (Merck) was added, and optical density (OD) was measured in a microplate reader (Bio-Rad, Hercules, CA, USA) at 405 nm. Each serum was also analysed for its binding profile against bEnd.3 proteins extracts by WB. Briefly, bEnd.3 total protein extracts, obtained after RIPA cells lysis buffer (50 Mm tris-HCl Ph 7.4; 150 Mm NaCl; 1% NP-40, 0.25% Na-deoxycholate), were separated by 11% SDS-PAGE and transferred into PVDF membranes as previously described [39]. Then, WB was performed with each serum at 1/500 dilution in PBS-BSA 1% followed by the goat-(alpha)-anti-rabbit IgG-Fc specific HRP. In addition, the BBB transmigration properties of each serum were evaluated in vitro and in vivo. For that, each serum was purified by protein A chromatography as previously described and then its BBB crossing properties were evaluated as described below in the in vitro BEB model and in vivo section.

### Example 18. Construction of Single-Domain Antibody Library

Total RNA was extracted from spleen and bone marrow of each rabbit using Trizol reagent (Invitrogen, Waltham, MA, USA) according to the manufacturer's instructions. First-strand Cdna was synthesized using the transcriptor first strand d Cdna synthesis kit (Roche, Basel, Switzerland). The first-strand cDNAs from each rabbit were then subjected to separate 30-cycle polymerase chain reactions using Phusion High Fidelity DNA polymerase (Thermo Fisher Scientific, Waltham, MA, USA) and 10 specific oligonucleotide primer combinations for the amplification of rabbit sdAbs in the format of the V_{L} (9×V_{κ} and 1×V_{λ}) as previously described [41, 42]. The PCR products were purified, digested with SfiI restriction enzyme (Roche), and cloned into the appropriately cut phagemid vector pComb3X [42, 43]. The recombinant phagemid was introduced into competent Escherichia coli ER2738 (Lucigen, Middleton, WI, USA) cells by electroporation and phages displaying the V_{L} sdAb library were produced as previously described and used immediately in the in vivo phage display panning.

### Example 19. In Vivo Phage Display

For the first selection round, three CD1 mice (Charles River, Wilmington, MA, USA) were intravenously injected into the tail vein with 100 μL of phages (˜1×10¹¹ phages/Ml) freshly prepared from the immune V_{L} sdAb library. For optimization purposes, phages were allowed to circulate for different time points (2 min, 60 min, 6 h or 24 h) and then the mice were sacrificed, perfused with PBS, and the brain extracted and weighted. Following brain homogenization in 70 μm cell strainers (VWR), cell homogenates were centrifuged at 1500×g at 4° C., 10 min. Then, the supernatant was discarded, the cell pellet resuspended in 2 Ml of wash buffer (PBS-0.05% Tween20), mixed with gentle agitation at room temperature for 2 min, and centrifuged at 1500×g at 4° C., 10 min. This wash step was repeated three times for the first selection round and five times for the next rounds. Then the phages were recovered by incubating the homogenized brain cells with 500 ML of freshly prepared trypsin (1 mg/Ml) (Gibco, Thermo Fisher Scientific)) supplemented with anti-protease (Merck) and DNAse (1 U/μL) (Invitrogen) for 15 min at 37° C. Then, the eluted phages were recovered after a centrifugation at 14,000×g for 10 min at 4° C. and normalized to a final volume of 1 Ml in PBS. Each output phage obtained was used for phage titration and re-amplification in ER2738 for a new round of in vivo selection. Phages were also tittered in blood. Three rounds of in vivo selections were performed for the 2 min and 60 min time points after a pilot study with all the time points described above. An irrelevant naïve rabbit V_{L} sdAb library and the M13 helper phage were also used as controls in the pilot study.

### Example 20. Analysis of In Vivo Phage Display Enrichment and Next Generation Sequencing

To analyse the enrichment and profile obtained after each round of in vivo phage display selection, individual clones from the initial V_{L} sdAb library, second and third selection rounds were randomly chosen and sequenced at Eurofins company (total of 64 clones). Sequence analysis was performed using the Vector NTI software (Invitrogen) and antibody frameworks, CDRs and the amino acid numbering and sequences alignment were performed as defined by Kabat et al. [44]. Furthermore, to analyse the overall diversity and enrichment obtained, we performed NGS. For that, we used the 250-paired ended module of the MiSeq (Illumina, San Diego, CA, USA) sequencing platform to obtain the whole sequence of the V_{L} sdAb regions selected in the third biopanning for both time points (2 min and 60 min). The Miseq library for DNA sequencing was prepared by amplifying the V_{L} sdAb regions and 2 μg of the purified amplicons were sent to the STABVIDA company for sequencing. The NGS sequence data was analysed using the Geneious software (Biomatters Ltd, Auckland, NZ). Sequence data was processed by merging the paired-ended sequence reads, translating the sequence into protein and discarding all sequences with less than 100 amino acids and no SfiI cut site and histidine tail sections. Subsequently, an in-house custom python script was developed to summarize and count the sequence reads. Bar charts representing the pattern of sequence reads were generated.

### Example 21. Expression and Purification of V_{L} sdAbs

To express and purify the selected clones, genes encoding the V_{L} sdAbs were transferred into the Pet21a plasmid (Novagen, Birmingham, UK) and transformed into Escherichia coli BL21 (DE3) electrocompetent cells (Invitrogen). A fresh colony of each V_{L} sdAb clone was grown overnight at 37° C. in Super Broth (SB) medium containing 100 μg/Ml of ampicillin. A 10 Ml sample of cells was used to inoculate one litre of SB medium containing 100 μg/Ml of ampicillin. Cells were grown at 37° C. until O.D._{600 nm}=0.6, induced with 0.6 Mm IPTG and growth was continued for 18 h at 19° C. After induction, bacteria were harvested by centrifugation (4000×g, 4° C., 15 min) and suspended in 50 Ml equilibration buffer (20 Mm NaH₂PO₄, 500 Mm NaCl, 30 Mm imidazole, and Ph 7.4) supplemented with protease inhibitors (Merck). Cells were lysed by sonication. Centrifugation (14,000×g, 4° C., 30 min) was used to remove cellular debris, and the supernatant was filtered through a 0.2 μm syringe filter. The V_{L} sdAbs were then purified by immobilized metal affinity chromatography (IMAC), using HP Histrap columns and the AKTA Start system (GE Healthcare, Chicago, IL, USA), using the C-terminal Hiss of Pet21a. After a washing step, elution of the V_{L} sdAbs occurred by a linear imidazole gradient from 60 to 300 Mm in elution buffer. The eluted fractions were pooled, desalted ANd concentrated in PBS using 3K Amicon columns (Merck). Then, the V_{L} sdAbs samples were loaded onto a HiPrep 16/60 Sephacryl S-100 HR gel filtration column (GE Healthcare) and pooled fractions were analysed for protein purity by 15% SDS-PAGE followed by Coomassie blue staining and WB with HRP-conjugated anti-His antibody (Roche). The concentration of proteins was determined by measuring the absorbance at 280 nm in the Nanodrop 2000 (Thermo Fisher Scientific). The same procedure was performed to express and purify the control antibody, FC5 VHH.

### Example 22. In Vitro BEB Models

The in vitro BEB models were optimized based on previous studies [45]. Briefly, bEnd.3 cells were cultured in DMEM, supplemented with 10% FBS and 1% penicillin/streptomycin (Lonza, Basel, Switzerland) antibiotic solution. Cells were cultured in a humidified atmosphere of 5% CO₂ at 37° C. and the medium changed every other day. The cells were adherent in monolayers and when confluent, harvested from cell culture flasks with trypsin EDTA (Gibco) and 4×10³ cells/well were seeded in 24-well plates tissue culture inserts (Falcon, Atlanta, GA, USA) previously coated with bovine plasma fibronectin (1 mg/Ml) (Merck). To allow the formation of tight junction's cells were incubated for 11-14 days and the media changed every 2 days. To evaluate the integrity of the in vitro BEB model before and during the assay day, a fluorescent probe of fluorescein Isothiocyanate-dextran with a MW of 4 kDa (FD4) and 40 kDa (FD40) (Merck) and stock concentration of 25 mg/Ml were diluted in transport buffer (TB) (5 Mm glucose, 5 Mm MgCl₂, 10 Mm HEPES at Ph 7.4 and 0.05% BSA) to an O.D._{493 nm} Of 0.1. Probes were then added to the apical side (apex) of the transwell and incubated for 2 h. Samples were recovered from the apex and base and fluorescence intensity was measured in a microtiter plate reader (BMG Labtech, Fluostar OPTIMA, Ortenberg, Germany) with an excitation of 485 nm and a maximum emission at 520 nm.

### Example 23. In Vitro BEB Translocation

To determine the in vitro BEB translocation efficiency of the selected V_{L} sdAb, 15 μg of each purified antibody was added to the apex of the transwell and incubated for 15 and 90 min. The incubation time and sdAb concentration were selected based on previous optimization assays (data not shown). Following incubation, the cells were washed once with PBS and three times with TB. Evaluation of the translocation efficiency of each V_{L} sdAb was assessed by running 15 Ml of the recovered volume from the apex and the base on a 15% SDS-PAGE acrylamide gel followed by WB analysis with HRP-conjugated anti-His antibody at 1:3000 (Roche). As a positive control, 100 ng of each purified V_{L} sdAb was used. The FC5 VHH was used as a positive control. The same procedure was performed to evaluate the BEB crossing properties of each purified rabbit serum, but the incubation was performed for 15 and 60 min and the detection was performed with goat-α anti-rabbit IgG-Fc specific HRP at 1:10,000. Chemiluminescence was detected using the Chemidoc XRS+System (Bio-rad).

### Example 24. Biodistribution Studies

The V sdAbs selected to proceed to in vivo biodistribution studies were radiolabelled with the radioactive precursor [^{99m}Tc(CO)₃(H₂O)₃]⁺, which was prepared by addition of a 0.9% saline solution of Na [^{99m}TcO₄], eluted from a ⁹⁹Mo/^{99m}Tc generator, to an IsoLink® kit (Covidien), according to Cantante et al. (Cantante, C.; Lourenço, S.; Morais, M.; Leandro, J.; Gano, L.; Silva, N.; Leandro, P.; 858 Serrano, M.; Henriques, A. O.; Andre, A.; Cunha-Santos, C.; Fontes, C.; Correia, J. D. G.; 859 Aires-da-Silva, F.; Goncalves, J. Albumin-Binding Domain from Streptococcus 860 Zooepidemicus Protein Zag as a Novel Strategy to Improve the Half-Life of Therapeutic 861 Proteins. J. Biotechnol. 2017, 253, 23-33. https://doi.org/10.1016/j.jbiotec.2017.05.017.). The radiochemical purity of the precursor was monitored by reversed-phase high-performance liquid chromatography (RP-HPLC) and instant thin-layer chromatography silica gel (ITLC-SG, Agilent Technologies, Santa Clara, CA, USA). Briefly, a specific volume of the fac-[^{99m}Tc(CO)₃(H₂O)₃]⁺ solution was added to a nitrogen-purged closed glass vial containing a solution of the His-tag containing sdAb in order to get a final concentration of 1 mg/Ml. The mixture reacted for 45-60 min at 37° C. and the radiochemical purity of ^{99m}Tc(CO)₃-sdAb was evaluated by ITLC-SG analysis using a 5% HCl (6 M) solution in MeOH as eluent. The precursors [^{99m}Tc(CO)₃(H₂O)₃]⁺ and [TcO₄]⁺ migrate in the front of the solvent (Rf=1), whereas the radioactive sdAb ^{99m}Tc(CO)₃-sdAb remains at the origin (Rf=0). Radioactivity distribution on the ITLC-SG strips was monitored using a miniGita Star scanning device (Raytest, Straubenhardt, DE) coupled with a Gamma BGO-V-Detector (Elysia Raytest, Straubenhardt, Germany). Purification of the ^{99m}Tc-labeled sdAb was performed using a 10 K Amicon (Merck Millipore) centrifugal filters for protein purification and concentration as described by the supplier. The filtrate was discarded and the concentrate containing ^{99m}Tc(CO)₃-sdAb was diluted in PBS and used for the biodistribution studies in CD1 mice. The radiochemical purity (>95%) was determined by ITLC-SG. Biodistribution studies of radiolabelled sdAbs were performed as previously described [31, 45]. Animals were intravenously injected into tail vein with the corresponding ^{99m}Tc(CO)₃-sdAb (0.2-7.9 MBq) diluted in 100 Ml of PBS Ph 7.2. Mice were sacrificed by cervical dislocation at 2 and 60 min after injection. The dose administered and the radioactivity in the sacrificed animals was measured using a dose calibrator (Carpintec CRC-15W). The difference between the radioactivity in the injected and the euthanized animals was assumed to be due to excretion. Brain and tissues of interest were dissected, rinsed in PBS to remove excess blood, weighed, and their radioactivity measured using a Y-counter (Berthold, Bad Wildbad, Germany). The uptake was calculated and expressed as a percentage of injected radioactivity dose per gram of organ or tissue (% ID/g).

### Example 25. Measurement of Brain Antibody Concentrations

To validate the in vivo translocation efficiency of each rabbit serum CD1 female mice were injected intravenously in the tail vein with 100 μg of purified antibody. Mice were sacrificed at 2 and 60 min after injection. The incubation time and antibody concentration were selected based on previous optimization assays (data not shown). Following blood recovery, mouse brain, kidney, and liver were isolated and homogenized as described above. The antibodies were recovered from each organ by immunoprecipitation (IP) with Dynabeads protein A pull-down beads (rabbit serum) according to the manufacturer protocol. 15 Ml of the IP elution was separated in 15% SDS PAGE gel and WB was performed with 1:3000 diluted conjugated 1:10,000 diluted anti-rabbit-HRP antibody. Chemiluminescence was detected using the Chemidoc XRS+System (Bio-Rad).

### Example 26. Development of PAN RG3 and FC5 Functionalized Liposomes

The encapsulation of PAN in liposomes was performed by an active loading method with an ammonium sulphate gradient as previously described by Chen et al. (Chen, R.; Zhang, M.; Zhou, Y.; Guo, W.; Yi, M.; Zhang, Z.; Ding, Y.; Wang, Y. The 912 Application of Histone Deacetylases Inhibitors in Glioblastoma. J Exp Clin Cancer Res 913 2020, 39. https://doi.org/10.1186/s13046-020-01643-6.).

Briefly, the relevant lipids, Dipalmitoyl phosphatidyl choline (DPPC), poly(ethylene glycol) (PEG-2000) covalently linked to distearoyl phosphatidyl ethanolamine (DSPE-PEG), and the functionalized DSPE-PEG phospholipid with biotin (DSPE-PEG-biotin), purchased from Avanti Polar Lipids, at a molar ratio of DPPC:Chol:DSPE-PEG:DSPE-PEG-biotin-1.85:1:0.14:0.01, were dissolved in chloroform and the organic solvent was removed by rotary evaporation. The formed homogeneous lipid film was hydrated with water and the so-formed suspension was frozen (−70° C.) and lyophilized in a freeze-dryer (Edwards, CO, USA) overnight. The rehydration of the lyophilized powder was performed with ammonium sulphate (135 Mm, Ph 5.4) at 45° C. for 30 min. In order to produce a homogeneous liposomal suspension, unloaded liposomes were filtered under nitrogen pressure (10-500 lb/in²), through polycarbonate membranes of proper pore size (at 45° C.), using a Lipex 57hermos-barrel extruder (Lipex: Biomembranes Inc., Vancouver, BC, Canada) until achieving liposomes with a mean size of around 0.1 μm. An ammonium sulphate gradient was created by replacing the extra liposomal medium with PBS buffer (Ph 7.4) using a Econo-pac 10 DG desalting column (Bio-Rad). PAN was incubated with unloaded liposomes, at a molar ratio 1:16 μmol of lipid, previously diluted in PBS (from a stock solution of 67 mg/Ml) for 60 min at 45° C. The non-encapsulated PAN was separated by ultracentrifugation at 250,000×g for 2 h at 15° C. in a Beckman LM-80 ultracentrifuge (Beckman Instruments, Inc., Fullerton, CA, USA). The pellet was suspended in PBS (Ph 7.4). RG3 and FC5 antibodies were biotinylated using the kit EZ-Link™ Sulpho-NHS-LC-Biotinylation Kit” (Thermo Fisher scientific), with a molar ratio of 1:30 (mole antibody:mole biotin). The biotin-antibody conjugate at was mixed with streptavidin a molar ration of 3:1 (mole antibody/mole streptavidin) at room temperature for 20 min. The mixture was then incubated with the pre-formed biotin-liposomes in a molar ratio of 1:1 (mole biotin in the liposome/mole antibody biotinylated) at room temperature for 2 h and later overnight at 4° C. Non-attached sdAb was removed by centrifugation using a 100 K Amicon® Ultra-4 membrane filter (Merck). Biodistribution studies of selected RG3-conjugated PAN liposomes were carried out with ¹¹¹In. For that, the chelating agent diethylenetriamine pentaacetic acid (DTPA) at a concentration of 6 μM was encapsulated during liposome preparation after achievement of the lipid film and before lyophilization [47]. RG3 functionalized liposomes co-loaded with DTPA were labelled with ¹¹¹In using the lipophilic complex ¹¹¹In-oxine as precursor. The ¹¹¹In-oxine complex passively crossed the lipid membrane and in the internal aqueous compartment of liposomes transferred the metal ion to DTPA where the hydrophilic complex remained 111 In-DTPA trapped. Radiolabelling and subsequent biodistribution studies were performed as described above.

### Example 27. Translocation Efficiency Assay of Lip-RG3 and Lip-FC5

Translocation efficiency of each liposome formulation was validated on the in vitro BEB model with bEnd.3 cells as described above. Briefly, empty rhodamine labelled RG3 and FC5 functionalized liposomes were previously diluted in DMEM without phenol red to a final concentration of 1.15 ng/Ml (considering the ratio of antibody) and added to the apical side of the in vitro BEB model. The apex volume and the base volume were collected after 90 min, 6 and 24 h, the fluorescence in those samples was measured separately in a microplate reader (Fluostar Optima Bmg Labtech) and the translocation was calculated using the equation:translocation (%)=Fi/Ft×100, where Fi is the recovered fluorescence intensity at the base and Ft is the fluorescence intensity of the total sdAb added to the apical side of the transwell. To determine the antitumour al effect of Lip-RG3 and Lip-FC5 encapsulated with PAN on LN229 cells, a cell viability assay was performed using the Cell Proliferation Reagent WST-1 (Roche). The cells were seeded at a density of 5×10³ cells/well in 96-well plates in 200 μL of DMEM culture medium supplemented with 10% FBS and 1% penicillin-streptomycin. The cells were subjected to increasing concentrations of PAN encapsulated liposomes, and the respective controls (free PAN, Lip-PAN, Lip-RG3, Lip-FC5 and empty liposome). After 24 h of treatment, WST-1 reagent was added, following the manufacturer's instructions, to determinate the cell viability. O. D._{450 nm} was measured in a plate reader following a 24 h incubation with the reagent. Each data point was determined using three replicate wells and two independent experiments. Best-fit IC₅₀ values were calculated using GraphPad Prism software (version 9.00; San Diego, CA, USA), using the log (inhibitor) vs. response (variable slope) function.

### Example 28. BEB-Glioblastoma In Vitro Model

To study the BEB translocation efficiency of the Lip-PAN-RG3 and Lip-PAN-FC5 liposomes and subsequent drug delivery and cytotoxic activity of PAN in a glioblastoma cell line, a dual in vitro non-contact co-culture model was established with bEnd.3 and LN229 cells. Briefly, bEnd.3 cells were cultured in tissue culture inserts until confluence as described above. 24 h before the assay day, LN229 cells were cultured in a 24-well plate at a density of 2×10⁴ in DMEM medium and the transwell bEnd.3 cells culture were transferred to the 24-well plates. Free and encapsulated PAN loaded RG3 and FC5 functionalized liposomes were added to the apical side of the transwell and incubated for 24 h. Following incubation all volume was recovered from the apex, the transwell was removed and the LN229 cells were incubated further 24 h. Following the 24 h treatment, WST-1 was added to the plate, and O.D._{450 nm} was measured following 24 h incubation. Each data point was determined using three replicate wells and two independent experiments were carried out in different days. To confirm that the cytotoxic effects of the BBB targeted PAN liposomes on GBM cell line was related to histone acetylation induction, the protein extract samples were quantified using the Bradford method (Coomassie Plus™ Kit, Thermo Fisher Scientific) according to the manufacturer's instructions and analysed by WB using anti-acetyl histone H3 (Lys9, Lys14) antibody (polyclonal, rabbit, 1:2500 dilution, Thermo Fisher Scientific), anti-histone H3 (polyclonal, rabbit, 1:1000 dilution, Thermo Fisher Scientific) as primary antibodies and anti-rabbit IgG-Fc specific HRP (polyclonal, goat, 1:10,000 dilution, Jackson ImmunoResearch) as secondary antibody. Protein detection was performed by chemiluminescence using Luminata Forte Western HRP (Merck) and acquired using the ChemiDoc XRS+ imaging system (Bio-Rad). In parallel, the integrity of the BEB model was measured as described previously.

### Example 29. Statistical Analysis

All data was expressed as mean±standard error of mean (SEM). Analysis was performed using Prism 9 (Graphpad Software). For in vitro assays, statistical significance of results was determined by One-way ANOVA followed by Tukey Multiple Comparison test to compare individual groups. Statistical analysis of the biodistribution data (ANOVA for evaluation of data from labelled sdAbs and t-test for data from liposomes) was also done with GraphPad Prism (version 9.00) and the level of significance was set as p-value<0.05.

1. An antibody-drug conjugated (ADC) for drug delivery with anti-tumour properties comprising a single domain antibody (sdAb), conjugated with an anti-tumour payload agent, wherein payload conjugation is in the free exposed cysteine at position 80, 23 or 88 of the VL unit and the single domain antibody (sdAb) is a VL unit engineered to exhibit a single free cysteine, said VL unit comprises rabbit sdAbs having at least a sequence selected from SEQ. ID. 1 to SEQ. ID. 43.

2. An antibody-drug conjugated (ADC) according to claim 1, wherein the VL chain comprises rabbit sdAbs having at least a sequence selected from SEQ. ID. 1, SEQ. ID. 2, SEQ. ID. 3, SEQ. ID. 4, SEQ. ID. 5, SEQ. ID. 6.

3. An antibody-drug conjugated (ADC) according to claim 1, wherein the anti-tumour payload agent is the SN38 molecule.

4. An antibody-drug conjugated (ADC) according to claim 1, wherein the anti-tumour payload agent SN38 molecule further comprises a maleimide, bonded through a diazaborine bioconjugation linker.

5. A pharmaceutical composition comprising an antibody-drug conjugated (ADC) as described in claim 1, further comprising an acceptable pharmaceutical agent.

6. A pharmaceutical composition according to claim 5 for use as a medicament in tumour therapies.

7. A pharmaceutical composition according to claim 6, wherein the tumour is a solid tumour, a hematopoietic or lymphoid tumour, preferentially the tumour is breast cancer, triple negative breast cancer, non-Hodgkin lymphoma, Hodgkin lymphoma, gastrointestinal cancer, acute myeloid leukaemia, multiple myeloma, cervical cancer, lung cancer, prostate cancer, colorectal cancer, ovarian cancer, kidney cancer, or thyroid cancer.

8. A pharmaceutical composition according to claim 6, wherein the tumour is located in a mammal, preferably is a human or canine tumour.

9. A process for obtaining an antibody-drug conjugated (ADC) comprising the following steps:
a) Providing lymph node primary cells derived from a canine multicentric lymphoma biobank
b) Rabbit immunization with 1×10⁷ of lymph node primary cells derived from a canine multicentric lymphoma biobank,
c) Isolation of RNA and cDNA from samples of spleen and bone marrow,
d) Construction of a single domain antibodies sdAB targeting cNHL and hNHL, as described in any of the claim 1 or 2,
e) Synthesis of rabbit sdAbs in the format of a VL light chain variable region with the free exposed cysteine at position 80, 23 or 88 of the V_{L} framework, as described in any of the claim 1 or 2
f) Binding of VLs to cNHL and hNHL cells with incubation of anti-HA FITC antibody,
g) Bioconjugation of VL with DAB-SN38 by adding a solution of DAB-SN38 to a solution of VL with TCEP.

10. A drug delivery system for targeting the BBB endothelial cell receptors of the central nervous system, comprising rabbit derived single-domain antibodies (sdAb) conjugated at the surface of liposomes encapsulated with a suitable drug enabling an efficient blood-brain barrier (BBB) translocation, wherein the sdAbs are defined by SEQ. ID. No. 44 to SEQ. ID. 108.

11. A drug delivery system according to claim 10, wherein the sdAb are defined by SEQ. ID. No. 44, SEQ. ID. No. 45, SEQ. ID. No. 46, SEQ. ID. No. 47, SEQ. ID. No. 48, respectively RG3, RG7, RG15, RG22 and RG23.

12. A drug delivery system according to claim 10, wherein the liposome having a sdAb conjugated at its surface is SEQ. ID. No. 44, SEQ. ID. No. 45, SEQ. ID. No. 46, SEQ. ID. No. 47, SEQ. ID. No. 48.

13. A drug delivery system according to claim 10, wherein the suitable drug is the pan-histone deacetylase inhibitor (PAN).

14. A pharmaceutical composition comprising a drug delivery system as described in claim 10, further comprising an acceptable pharmaceutical agent.

15. A process for the production of a drug delivery system as described in claim 10, comprising the following steps:
a) Providing immunized rabbits with a cell line of mouse brain endothelial cells (bEnd.3),
b) Recovery of rabbit antibodies capable of transpose the BBB barrier from the sera of immunized rabbits as described in a),
c) Providing a sdAb library wherein the sdAb are from the antibody light chain variable regions (V_{L}) recovered from the bone marrow and spleen cDNA of the immunized rabbits, and cloned said V_{L} sdAbs regions in the pComb3X phagemid vector,
d) Selecting the sdAbs displayed at phage surface with targeting specificity properties of blood-brain barrier (BBB) translocation,
e) Conjugation of a sdAbs at the surface of liposomes,
f) Encapsulation of a suitable drug enabling an efficient blood-brain barrier (BBB) translocation to the conjugated liposomes of e).

16. A process according to claim 15, wherein the sdAbs of d) are further treated by injecting a sdAbs composition in CD1 mice and recovering the phages from the mice brain to retrieve brain specific sdAbs.

17. A process according to claim 16 wherein the treatment is repeated one time, preferably two times, even preferably three times, thus resulting of enrichment phages having a titter of 10⁵ (phages/mL), which present improved ability to reach the BBB.
